SEQUENCE LISTING (8 sequences)

SEQ ID NO: 1 - DNA, 21 bp, synthetic construct
  misc_feature 1..21: note = FANCD2D forward primer
  source 1..21: mol_type = other DNA
acctgttatgagcgtgaagtc

SEQ ID NO: 2 - DNA, 21 bp, synthetic construct
  misc_feature 1..21: note = FANCD2D reverse primer
  source 1..21: mol_type = other DNA
gatgcaggactgtgcattaga

SEQ ID NO: 3 - DNA, 21 bp, synthetic construct
  misc_feature 1..21: note = EXD2 forward primer
  source 1..21: mol_type = other DNA
ggtctggcctaaggtttcttc

SEQ ID NO: 4 - DNA, 19 bp, synthetic construct
  misc_feature 1..19: note = EXD2 reverse primer
  source 1..19: mol_type = other DNA
cagttcacgctgggttctt

SEQ ID NO: 5 - DNA, 21 bp, synthetic construct
  misc_feature 1..21: note = XRCC forward primer
  source 1..21: mol_type = other DNA
gcagtcttcctagtctcaact

SEQ ID NO: 6 - DNA, 22 bp, synthetic construct
  misc_feature 1..22: note = XRCC reverse primer
  source 1..22: mol_type = other DNA
ttgcccttctaggagcttaatg

SEQ ID NO: 7 - DNA, 20 bp, synthetic construct
  misc_feature 1..20: note = exon 2 of SIK2
  source 1..20: mol_type = other DNA
aataatcgataagtctcagc

SEQ ID NO: 8 - DNA, 20 bp, synthetic construct
  misc_feature 1..20: note = exon 4 of SIK2
  source 1..20: mol_type = other DNA
gattttcagctttgaggtca

(19) United States
(12) Patent Application Publication
(10) Pub. No.: US 2024/0082232 A1
(43) Pub. Date: Mar. 14, 2024

(54) COMPOSITIONS AND METHODS FOR TREATMENT OF OVARIAN AND BREAST CANCER

(71) Applicant: Board of Regents, The University of Texas System, Austin, TX (US)

(72) Inventors: Zhen LU, Pearland, TX (US); Robert C. BAST, JR., Houston, TX (US)

(21) Appl. No.: 18/469,467

(22) Filed: Sep. 18, 2023

Related U.S. Application Data
(63) Continuation of application No. PCT/US2021/038571, filed on Jun. 23, 2021.
(60) Provisional application No. 63/164,308, filed on Mar. 22, 2021.
(60) Provisional application No. 63/163,118, filed on Mar. 19, 2021.

(51) Int. Cl. A61K 31/454; A61K 31/502; A61K 31/5025; A61P 35/00

(52) U.S. Cl. CPC A61K 31/454; A61K 31/502; A61K 31/5025; A61P 35/00

(57) ABSTRACT

Provided are methods of treating cancer comprising administering to a patient in need thereof a poly (ADP-ribose) polymerase (PARP) inhibitor and a salt-induced kinase 2 (SIK2) inhibitor. Also provided are methods for increasing the duration of remission for cancer, and for enhancing the sensitivity of PARP inhibitors for cancer.

This application is a bypass continuation of International Application No. PCT/US2021/038571, filed Jun. 23, 2021, which claims priority to, and the benefit of, U.S. Application No. 63/163,118, filed Mar. 19, 2021, and U.S. Application No. 63/164,308, filed Mar. 22, 2021, the entireties of each are incorporated by reference herein.

### GOVERNMENT LICENSE RIGHTS

This invention was made with government support under Grant Contract No. P50 CA217685 awarded by the National Institutes of Health. The government has certain rights in the invention.

### INCORPORATION OF SEQUENCE LISTING

The sequence listing that is contained in the file named “MDA0065-201BC1-US,” which is 10.9 kilobytes as measured in Microsoft Windows operating system and was created on Sep. 18, 2023, is filed electronically herewith and incorporated herein by reference.

Recent studies indicate that DNA damage, aberrations in the DNA damage response and defects in DNA repair machinery play a major role in ovarian and triple-negative breast cancer (TNBC). DNA double-strand breaks (DSBs) are considered one of the most cytotoxic forms of DNA damage that can lead to mutation and trigger permanent growth arrest or cell death. The two main DSB repair pathways include non-homologous end-joining (NHEJ) and homologous recombination (HR). NHEJ is a rapid, high-capacity pathway that joins two DNA ends using ligase IV/XRCC4 (X-Ray Repair Cross Complementing 4) complex that recognizes DSBs. NHEJ can, however, accommodate very limited base pairing between the two processed DNA ends, thereby potentially forming repair joints with up to four base pairs of ‘microhomology.’ By contrast, HR requires extensive sequence homology between the broken DNA and a donor DNA molecule. The end resection regulated by EXO1 (exonuclease 1) at DSBs and the DNA synthesis using intact homologous DNA sequence as templates are the key steps in the HR repair process. The Fanconi Anemia (FA) pathway is closely linked to HR repair through its functional interaction with BRCA1/2. FA-group D2 (FANCD2) protein promotes HR repair and prevents DNA DSB formation and chromosomal aberrations in DNA damaged cells. Most DNA repair pathways are complex, involving many proteins working in discrete consecutive steps. Therefore, the efficiency of DNA repair requires transcription factors controlling and maintaining the expression of DNA repair genes. DNA DSB repair is a critical prerequisite for cancer cell survival; it may also provide therapeutic opportunities.

There is a need for more effective therapies for ovarian cancer. Like with many other agents, resistance has been reported with PARP inhibitors including Olaparib. The side effect profile of new PARP inhibitors appears to be more tolerable than older chemotherapies, however PARP inhibitors do produce side effects in patients, such as nausea and constipation, as well as hematologic toxicities, including decreased white blood cells and red blood cells. As Compound B has shown synergy with PARP inhibitors and little or no hematopoietic toxicity in pre-clinical studies, it is a promising candidate for combination therapies for more effective treatment of ovarian and breast cancers, as well as for maintaining primary or subsequent remissions.

Provided is a method of increasing the magnitude and/or duration of activity of a poly (ADP-ribose) polymerase (PARP) inhibitor in a patient being treated with the PARP inhibitor, comprising administering to the patient in need thereof the PARP inhibitor and a salt-induced kinase 2 (SIK2) inhibitor.

Also provided is a method of increasing the sensitivity of cancer cells to treatment with a PARP inhibitor, comprising contacting the cells with the PARP inhibitor and a SIK2 inhibitor.

Also provided is a method of prolonging survival in a cancer patient in need thereof comprising administering to the patient in need thereof a PARP inhibitor and a SIK2 inhibitor.

Also provided is a method of suppressing tumor growth in a cancer patient in need thereof, comprising administering to the patient in need thereof a PARP inhibitor and a SIK2 inhibitor.

Also provided is a method of increasing the duration of remission for a cancer patient in need thereof comprising administering to the patient in need thereof a PARP inhibitor and a SIK2 inhibitor.

Also provided is a method of preventing a relapse or reducing the incidence of relapse of a cancer patient in remission, the method comprising administering to the patient in need thereof a PARP inhibitor and a SIK2 inhibitor.

Also provided is a method for reducing incidences of, or risk of, cancer recurrence in a patient deemed to be at risk of cancer recurrence, the method comprising administration to the subject of a PARP inhibitor and a SIK2 inhibitor.

Also provided herein is a method of reducing hematologic toxicity during cancer treatment comprising administering to a patient in need thereof a PARP inhibitor and a SIK2 inhibitor.

Also provided herein is a method of maintaining hematopoiesis during cancer treatment comprising administering to a patient in need thereof a PARP inhibitor and a SIK2 inhibitor.

Also provided herein is a method of reducing toxicity and maintaining hematopoiesis during cancer treatment comprising administering to a patient in need thereof a PARP inhibitor and a SIK2 inhibitor.

Also provided herein is a method of preventing hematologic side effects during cancer treatment comprising administering to a patient in need thereof a PARP inhibitor and a SIK2 inhibitor.

Each and every method, composition, or use described herein also optionally includes the limitation, “wherein the cancer is not ovarian cancer.” Each and every method, composition, or use described herein also optionally includes the limitation, “wherein the cancer is not ovarian cancer without BRCA1/2 mutations.” Each and every method, composition, or use described herein also optionally includes the limitation, “wherein the cancer is not a PARP inhibitor-resistant ovarian cancer.”

Each and every method, composition, or use described herein also optionally includes the limitation, “wherein the SIK2 inhibitor is not Compound A or Compound B.”

Each and every method, composition, or use described herein also optionally includes the limitation, “wherein the PARP inhibitor is not olaparib.”

Each and every method, composition, or use described herein also optionally includes the limitation “if the PARP inhibitor is Olaparib and the SIK2 inhibitor is Compound A or Compound B, then the cancer is not ovarian cancer.”

Each and every method, composition, or use described herein also optionally includes the limitation “if the PARP inhibitor is Olaparib and the SIK2 inhibitor is Compound A or Compound B, then the cancer is not breast cancer.”

These and other embodiments disclosed herein are described in detail below.

### BRIEF DESCRIPTION OF THE SEQUENCES

SEQ ID NO:1—FANCD2D forward primer.
SEQ ID NO:2—FANCD2D reverse primer.
SEQ ID NO:3—EXD2 forward primer.
SEQ ID NO:4—EXD2 reverse primer.
SEQ ID NO:5—XRCC forward primer.
SEQ ID NO:6—XRCC reverse primer.
SEQ ID NO:7—Sequence of exon 2 of SIK2 for CRISPR/Cas9 knockout in OVCAR8 and SKOv3 cell lines.
SEQ ID NO:8—Sequence of exon 4 of SIK2 for CRISPR/Cas9 knockout in OVCAR8 and SKOv3 cell lines.

### BRIEF DESCRIPTION OF THE DRAWINGS

FIG. 1—Shows that SIK2 inhibitors enhance olaparib sensitivity in ovarian cancer and breast cancer cells. (A) Dose-response curves for Compound A or Compound B (blue), olaparib (green) or Compound A or Compound B combined with olaparib (red) for 96 hrs in 12 cancer cell lines and 3 non-malignant cell lines. The IC₅₀s of inhibitors and concentration ratio of SIK2 inhibitors to olaparib used in each cell line are listed in Table 2. The statistical significance between olaparib alone and SIK2 inhibitor combined with olaparib was calculated with two-way ANOVA multiple comparisons. ***p<0.001, ****p<0.0001, ^{ns}p>0.05 (red stars indicate SIK2 inhibitor+olaparib enhancing the effect of olaparib alone; blue stars indicate SIK2 inhibitor+olaparib inhibiting olaparib's effect. A combination index (CI) at ED 90 was calculated using CalcuSyn software. Representative experiments were from two independent experiments with four technical repeats per experiment. (B) Dose-response curves of olaparib in paired cancer cell lines with or without knockout of SIK2 (top) and with or without stable transfection of SIK2 (bottom). The median inhibitory concertation (IC₅₀) of olaparib was calculated using GraphPad Prism 8. Representative experiments are from two independent experiments with four technical repeats per experiment. Western analysis confirmed either SIK2 knock out (top) or overexpression (bottom). (C) Representative images of clonogenic assays (top) and quantification of colonies (bottom) in four cancer cell lines are presented. SKOv3, OVCAR8, HCC5032, and MDA-MB-231 cells were treated with olaparib, Compound A, Compound B, or olaparib+Compound A or Compound B at concentrations indicated in FIG. 2A for 10-22 days. The columns indicate the mean of colonies and the bars indicate the S.D. (** p<0.01, *** p<0.001, ****p<0.0001). The data were obtained from three independent experiments.

FIG. 2—Shows that SIK2 inhibitors enhance rucaparib, olaparib, niraparib and talazoparib sensitivity in ovarian cancer. (A) Representative images of clonogenic assay in four cancer cell lines are presented (left). SKOv3, OVCAR8, HCC5032, and MDA-MB-231 cells were treated with olaparib, Compound A, Compound B alone, or olaparib plus Compound A or Compound B at concentrations indicated for 10-22 days (right). (B) Dose-response curves of Compound A/Compound B (blue), PARP inhibitors (rucaparib, olaparib, niraparib, or talazoparib) (green) or Compound A/Compound B combined with PARP inhibitor (red) for 96 hrs in OVCAR8 and SKOv3 ovarian cancer cells. Combination index (CI) was calculated using CalcuSyn software. Representative experiments were from two independent experiment and 4 technical repeats per experiment.

FIG. 3—Shows the effect of Compound A, Compound B, and olaparib on PARP1 enzyme activity and trapping. (A) PARP1Trapping in OVCAR8 and MDA-MB-231 cells. Cells were treated with Compound A, Compound B, olaparib alone, or olaparib+Compound A or Compound B for 72 hrs. The concentrations of Compound A, Compound B, and olaparib are 4 μM, 4 μM, and 6 μM, respectively. Western blot analysis of chromatin-bound fractions of PARP1. (B) Western blot analysis of PARP1 protein expression. (C) The dose-response effect of olaparib and SIK2 inhibitor on PARP1 enzyme activity. OVCAR8 and MDA-MB-231 cells were treated with SIK2 inhibitors for 26 hrs as indicated. The columns indicate the mean of activity and the bars indicate the S.D. (^{ns}p>0.05, ** p<0.01, ***p<0.001, ****p<0.0001). Representative experiments were from three independent experiments and 4 technical repeats per experiment.

FIG. 4—Shows the combined effect of SIK2 inhibitor and olaparib on PARP-1 enzyme activity and DNA DSB repair pathways. (A) Dose-response curves for olaparib (top) and combined effect of SIK2 inhibitors with olaparib on PARP-1 enzyme activity (bottom). OVCAR8 and MDA-MB-231 cells were treated with SIK2 inhibitors, olaparib alone, or the combination for 26 hrs. The concentrations of Compound A, Compound B, and olaparib are 6 μM, 4 μM, and 0.05 μM, respectively (also see FIG. 3B-C). The columns indicate the mean of activity and the bars indicate the S.D. (* p<0.05, ** p<0.01, ***p<0.001, ****p<0.0001). Representative data are from three independent experiments with 4 technical repeats per experiment. (B) Dose-response curves of Compound A, Compound B, and olaparib in DT40 PARP-1 −/− cells with and without knock-in of human PARP-1 (hPARP) (top and bottom left panels). The IC₅₀ indicated on the curves was calculated using GraphPad Prism 8. The expression of exogenous hPARP in DT40 PARP-1 −/− was measured by western blotting (bottom left panel). Representative data were from two independent experiments with 4 technical repeats per experiment. (C) The heatmap presentation of unsupervised hierarchical clustering of gene expression. The heatmap includes 3587 transcripts (up or down-regulated by ≥2-fold) treated with Compound A, Compound B, olaparib, Compound A+olaparib and Compound B+olaparib. The heatmap illustrates changes that are color coded with red corresponding to up-regulation and green to down-regulation. (D) The Venn representation. Venn diagram analysis represented the number of genes (up or down-regulated by ≥2-fold) were overlapped by the treatment of Compound A+olaparib (yellow) or Compound B+olaparib (green). (E) Go analysis of 1380 differentially expressed genes shared by Compound A+olaparib or Compound B+olaparib treatments. The bar plot shows the log₁₀P value of the biological process GO terms obtained with differentially expressed genes at p<0.01. Red highlights indicate biological processes involved in DNA damage and repair.

FIG. 5—Shows that Compound A and Compound B enhances olaparib-induced DNA DSBs and apoptosis. (A) The Heatmap representation unsupervised hierarchical clustering of differentially expressed genes associated with DNA repair. The heatmap contains changes that are color coded with red corresponding to up-regulation and green to down-regulation. (B) Analysis of DNA Repair and apoptosis genes. BRCA2, EXO1, FANCD2, LIG4, XRCC4, BAX, BCL2, CASP7, and TRADD were analyzed using RT-PCR in OVCAR8 ovarian and MDA-MB-231 breast cancer cells. Cells were treated with Compound A, Compound B, olaparib alone, or olaparib+Compound A or Compound B for 72 hrs. The concentrations of Compound A, Compound B, and olaparib are 4 μM (2 times), 4 μM (3 times), and 15 μM (2 times), respectively. The columns indicate the mean of RNA expression and the bars indicate the S.D. (* p<0.05, ** p<0.01, ***p<0.001, ****p<0.0001). Representative data are from two independent experiments with 3 technical repeats per experiment. (C) Quantification of DNA damage (γ-H2AX). Endogenous γ-H2AX was stained with anti-γ-H2AX antibody in the cells treated with single agent or combined for 8 hrs as indicated. The concentrations of Compound A, Compound B, and olaparib were 1 μM, 4 μM, and 2 μM, respectively. Red indicates γ-H2AX and Blue-DAPI indicates nuclear stain. Representative images are presented (right). Bar 20 γm. Red γ-H2AX dots were quantified with OLYMPUS CellSens Dimension software. The middle solid lines indicate the mean of fluorescent dots. The top and bottom solid lines indicate the S.D. (*** p<0.001, ****p<0.0001). (^{ns}p>0.05, ** p<0.01, ***p<0.001, ****p<0.0001) (left). Experiments were from three independent experiments with a total of 100-200 cells per treatment. Bar 20 μm. (D) Detection of apoptosis using Annexin V/Propidium iodide (PI) staining. SKOv3 cells were treated with Compound A (8 μM), Compound B (5 μM), olaparib (25 μM) alone or combined for 6 days as indicated. HCC5032 cells were treated with Compound A (1 μM), Compound A (3 μM) or olaparib (3 μM) alone or combined for 5 days. OVCAR8 and MDA-MB231 were treated with treated with Compound A (6 μM), Compound B (6 μM) or olaparib (5 μM) alone or combined for 5 days. The columns indicate the mean of Annexin V positive cells and the bars indicate the S.D. (* p<0.05, ** p<0.01, ***p<0.001, ****p<0.0001). Representative data are from three independent experiments with 3 technical repeats per experiment.

FIG. 6—Shows that Compound A and Compound B enhance olaparib-induced DNA double-strain breaks and apoptosis. (A) The heatmap of unsupervised hierarchical clustering of differently expressed genes associated with apoptosis. The map contains changes that are color coded with red corresponding to up-regulation and green to down-regulation. (B) Analysis of DNA repair and apoptosis gene. BRCA2, EXO1, FANCD2, LIG4, XRCC4, BAX, BCL2, CASP7, and TRADD were analyzed using RT-qPCR in SKOv3 and OVCAR8 ovarian cancer cells. Cells were treated with Compound A, Compound B, olaparib alone, or olaparib+Compound A or Compound B for 72 hrs. The concentrations of Compound A, Compound B, and olaparib are 4 μM (2 times), 4 μM (3 times), and 15 μM (2 times), respectively. The columns indicate the mean of RNA expression and the bars indicate the S.D. (* p<0.05, ** p<0.01, ***p<0.001, ****p<0.0001). Representative experiments were from two independent experiment and 3 technical repeats per experiments. (C) Quantification of DNA damage using comet assay. Cells were plated and treated with Compound A, Compound B, or olaparib on the comet slides for total 48 hrs and with and without olaparib for 16 hrs (16 hrs before harvest). 1 μM of Compound A was applied to HCC5032, OVCAR8, and SKOv3 and 0.5 μM to MDA-MB-231 cells. 4 μM of Compound B and 5 μM of olaparib are applied to all 4 cell lines tested. Slides were then stained with Vista Green DNA dye and viewed using Olympus fluorescence microscope with FITC filter. Olive Tail Moment was measured using CaspLab1.2.3β2 software. The columns indicate the mean of tail moments and the bars indicate the S.D. (* p<0.05, ** p<0.01, *** p<0.001, **** p<0.0001). Representative experiments were from three independent experiments with a minimum of 50 cells.

FIG. 7—Shows that Compound A and Compound B decrease phosphorylation of HDAC4/5/7 and promoter activity of MEF2 transcription factors. (A) Phosphorylation level of HDAC4/5/7. Twenty-one ovarian and one triple-negative breast cancer cell lines were treated with Compound A (4 μM) (top panel) or Compound B (4 μM) (bottom panel) for 24 hrs. Western blots were probed with specific antibodies as indicated. (B) Detection of HDAC5 localization with or without SIK2 inhibitors. OVCAR8 and MDA-MB-231 cells were plated on 2-well chamber slides. After overnight incubation, cells were treated with Compound A (3 μM) or Compound B (5 μM) for 24 hrs. Cells were stained with anti-HDAC5 and imaged with fluorescence microscopy for HDAC5 (green) and DAPI nuclear stains (blue). The fluorescent intensity of nuclear HDAC5 was quantified using ImageJ (FIG. 8). The bar represents 20 gm. Data were from three independent experiments with a total of 100-200 cells per group. (C) Quantification of MEF2 promoter activity. Cells were plated and incubated overnight. Cells were then transfected with a mixture of a MEF2-responsive luciferase construct and a constitutively expressing Renilla luciferase construct (40:1) for 24 hrs. Cells were re-plated into 96 well plates and then treated with Compound A (4 μM) and Compound B (4 μM) for different intervals or with different doses of Compound A and Compound B for 24 hrs as indicated. Cells were lysed for dual luciferase assays. The relative luciferase activity of MEF2 was calculated by normalizing to Renilla luciferase activity. The columns indicate the mean of MEF2 luciferase activity and the bars indicate the S.D. (^{ns}p>0.05, * p<0.05, ** p<0.01, *** p<0.001, **** p<0.0001). Representative data were from two independent experiments with 3 technical repeats per experiment. (D) Quantification of MEF2 promoter activity with and without knockdown of HDAC4 and HDAC5. Cells were transfected with targeting or control siRNA for 24 hrs prior to transfection of a mixture of a MEF2-responsive luciferase construct and Renilla luciferase construct. Cells were re-plated into 96 well plates and then treated with Compound A (4 μM) or Compound B (4 μM) for 24 hrs. Luciferase activity was measured and analyzed as described in (C) (top panel). HDAC4 and HDAC5 siRNA knockdown efficiency was measured by western blot analysis (bottom panel). Representative data are from two independent experiments.

FIG. 8—Shows that Compound A and Compound B decrease phosphorylation of HDAC4/5/7 and promoter activity of MEF2 transcription factors. (A) Detection of HDAC5 localization with and without SIK2 inhibitors using immunofluorescence staining Nuclear fluorescent intensity was measured by ImageJ (related to FIG. 7B). Experiments were from three independent experiments with total 100-200 cells per group. The middle solid lines indicate the mean of fluorescent intensity. To top and bottom solid lines indicate the S.D. (*** p<0.001, ****p<0.0001). (B) Detection of HDAC5 localization with and without SIK2 inhibitors using cell fractionation. OVCAR8 and MDA-MB-231 cells were treated with Compound A (6 μM) or Compound B (5 μM) for 26 hrs. Total cell lyses were collected for cell fractionation and cytoplasmic extracts and nuclear extracts were subjected to western analysis using the antibodies indicated. (D and L indicate dark and light exposure, respectively). (C) Quantification of MEF2 promoter activity (related to FIG. 7C). Cells were plated and after overnight incubation, then transfected with a mixture of a MEF2-responsive luciferase construct and a constitutively expressing Renilla luciferase construct (40:1) (QIAGEN) for 24 hrs. Cells were re-plated into 96 well plate and then treated with olaparib (4 μM) for different time intervals or with different doses of olaparib for 24 hrs as indicated. Cells were lysed for dual luciferase assay. The relative luciferase activity of MEF2 was calculated by normalizing to Renilla luciferase activity. The columns indicate the mean of MEF2 luciferase activity and the bars indicate the S.D. (^{ns}p>0.05, * p<0.05). Representative experiments were from two independent experiments and 3 technical repeats per experiment. (D) Quantification of MEF2 promoter activity (related to FIG. 7D). Cells were treated with TMP195 for 24 hrs prior to transfection of a mixture of a MEF2-responsive luciferase construct and Renilla luciferase construct. Cells were re-plated into 96 well plate and then treated with Compound A (4 μM) and Compound B (4 μM) for 24 hrs. Measurement of luciferase activity is performed, quantified, and analyzed as described in (C) (top panel). The bars indicate the S.D. (^{ns}>0.05, * p<0.05, ****p<0.0001). Representative experiments were from two independent experiments and 3 technical repeats per experiment. (E) Working model. SIK2 inhibitor inhibits class IIa HDAC/MEF2D-mediated downregulation of genes that are associated with DNA repair.

FIG. 9—Shows that SIK2 inhibition alters MEF2D transcription factor-mediated downstream signaling. (A) Alterations affecting MEF2 family genes in ovarian and breast cancer by TCGA analysis. Alterations of MEF2D are found in 12% of ovarian cancer samples (TCGA, 316 samples, Nature 2011) and 26% of breast cancer samples (Metabric, 2509 samples, Nature 2012 & Nat Commun 2016), respectively, and the large majority of alterations were amplifications and mRNA upregulations. Data and plots were obtained using cBioPortal (21, 58, 59). (B) MEF2D consensus DNA motifs. The MEF2 motif is enriched in MEF2D-binding sites in SKOv3 cells. (C) GO analysis of MEF2D-bound genes. (D) Chip sequence of anti-MEF2D at the FANCD2 locus in SKOv3 cells treated with and without Compound A. The dotted line indicates the comparison of chromatin accessibility of the FANCD2 gene between control and Compound A treatment. (E) Chip and RT-qPCR analysis of FANCD2, EXO1, and XRCC4 genes. OVCAR8 and MDA-MB-231 cells were treated with and without Compound A (6 μM) or Compound B (4 μM) for 48-50 hrs and then harvested subjecting to Chip with normal IgG, MEF2D, Pol-II, H3K27Ac, or H3KMe1 antibody as indicated. Chip pull-down samples were analyzed by RT-qPCR. The columns indicate the mean of relative fold changes (Fold change=2-DDCt, Chip signal relative to the IgG background signal) and the bars indicate the S.D. (* p<0.05, ** p<0.01, *** p<0.001, **** p<0.0001). Representative data are from two independent experiments and 3 technical repeats per experiment.

FIG. 10—Shows that SIK2 inhibition alters MEF2D transcription factor-mediated downstream signaling (related to FIG. 9). (A) MEF2D-binding sites in human ovarian cancer cells. (B) Chip analysis of FANCD2, EXO1, and XRCC4 genes. SKOv3, SKOv3- and OVCA8-SIK2 knockout cells were treated with and without Compound A (6 μM) or Compound B (4 μM) for 48-50 hrs and then harvested subjecting to Chip with normal IgG, MEF2D, Pol-II, H3K27Ac, or H3KMe1 antibody as indicated. Chip pull-down samples were analyzed by RT-qPCR using as indicated. The columns indicate the mean of relative fold changes (Fold change=2-DDCt, Chip signal relative to the IgG background signal) and the bars indicate the S.D. (* p<0.05, ** p<0.01, *** p<0.001, **** p<0.0001). Representative experiments were from two independent experiments and 3 technical repeats per experiment.

FIG. 11—Shows clinical data analysis by log-rank test (gepia.cancer-pku.cn/). Kaplan Meier survival curves of FANCD2, EXO1, and XRCC1 in ovarian and breast cancer.

FIG. 12—Shows that overexpression of MEF2D is sufficient to block SIK2 inhibition-induced downregulation of FANCD2, EXO1, and XRCC4, DNA damage and growth inhibition. (A) Forced expression of MEFD2. DOX-Inducible MEF2D expression OVCAR8 and MDA-MB-231 cells were treated with Compound A (1 μM), Compound B (4 μM), and olaparib (2 μM) in present and absent of DOX (1 μg/ml) for 8 hrs. DOX was added to culture medium 48 hrs prior to inhibitor treatments. Red indicates γ-H2AX and Blue-DAPI indicates nuclear stains. Reprehensive images were presented (Left). Bar 20 μm. Red γ-H2AX dots were quantified with OLYMPUS CellSens Dimension software. The middle solid lines indicate the mean of fluorescent dots. To top and bottom solid lines indicate the S.D. (^{ns}p>0.05, ** p<0.01, ***p<0.001, **** p<0.0001) (right). Bar 20 μm. Representative experiments were from two independent experiments and 3 technical repeats per experiment. (B) Determination of MEF2D expression by western analysis. (C) Determination of cell viability in MEF2D DOX-Inducible OVCAR8 and MDA-MB-231 cells. DOX-Inducible MEF2D sublines of OVCAR8 and MDA-MB-231 were treated with DOX and without DOX for 24 hrs, and then treat with Compound A (2 μM), Compound B (4 μM), and olaparib (4 μM) for 72 hrs. The statistical significance between DOX- and DOX+ was calculated with one-way ANOVA multiple comparisons. ****p<0.0001, ^{ns}p>0.05. Representative data are from three independent experiments with 4 technical repeats per experiment.

FIG. 13—Shows that co-administration of SIK2 inhibitor and olaparib synergistically inhibits xenograft growth. (A) Tumor growth and (B) Tumor weight of ovarian cancer xenografts in female athymic nu/nu mice after treatment with Compound A, Compound B, olaparib, Compound A+olaparib, and Compound B+olaparib. SKOv3 (5×10⁶) or OVCAR8 (3×10⁶) cells were injected subcutaneously (sub-q) or intraperitoneally (ip). After 7-day inoculation, mice (n=8-10) were treated with Compound A, Compound B, olaparib, or combination as indicated by gavage 5 days per week for 4-6 weeks. Tumor growth by tumor volume (A) or tumor weight (B)under different treatments was plotted as mean±S.D. (*p<0.05; ** p<0.01). (C) Tumor weight of ovarian cancer cells in female athymic nu/nu mice after treatment with Compound B, olaparib, and Compound B+olaparib. 3.5×10⁶ OC316 tumor cells were injected i.p. On day 7 after inoculation, mice (n=20) were treated with Compound A, Compound B, olaparib, or a combination as indicated by gavage 5 days per week for 5 weeks. Mice with ip tumor growth (n=10 mice per group) were sacrificed and tumors were weighed after completing 5 weeks of treatment. Tumor growth by weight under different treatments was plotted as mean±S.D. (*p<0.05; ** p<0.01). Survival (ethical endpoint) of the remaining 10 mice per group was evaluated. Survival curves were generated by GraphPad Prism 6. (^{ns}p>0.05, *p<0.05; ** p<0.01). (C) Tumor growth of MDA-MB-231 breast cancer cell in female athymic nu/nu mice. 0.8×10⁶ MDA-MB-231 cells were injected into the fourth mammary fat pads. Tumor-bearing mice were randomized into 4 treatment groups (n=10) after 7-days of tumor growth. Mice were treated with Compound B, olaparib, and Compound B+olaparib for 5 weeks as indicated. Tumor growth was measured and survival (ethical endpoint) was evaluated from the start of treatment until tumors reached 1000 mm³. Survival curves were generated by GraphPad Prism 6. (^{ns}p>0.05, *p<0.05; ** p<0.01). (E) Representative images of IHC with indicated antibodies from OVCAR8 and MDA-MB-231 mouse tumor tissues. Scale bar, 50 μM. Positive cells per one hundred cancer cells were counted and analyzed using GraphPad Prism 8 (^{ns}p>0.05, ** p<0.01, ***p<0.001, ****p<0.0001). #1 indicates mouse #1 and #2 indicates mouse #2.

FIG. 14—Shows that co-administration of SIK2 inhibitor and olaparib is synergistic in vivo (related to FIG. 13). Both mice body weights and ascites volume of OVCAR8 (A) and OC316 (B) intraperitoneal models were evaluated after end of experiments. The middle solid lines indicate the mean of ascites volume (left) or body weight (right). (^{ns}p>0.05, ** p<0.01, *** p<0.001).

FIG. 15—Shows SIK2 expression in the breast cancer tissue and cell lines. (A) Immunohistochemistry staining of TMA and analysis of SIK2 expression in the bar graph and (B) SIK2 expression in breast cancer cell lines by western blot analysis.

FIG. 16—Shows that Compound B enhances paclitaxel sensitivity. (A) Compound B inhibits organoid growth, inducing cell death. (B) SIK2 expression is inversely correlated with SIK2 expression, p=0.0277. (C) Compound B enhanced paclitaxel sensitivity, inhibiting growth of MDA-MB-231 xenografts (top) and prolonging survival of mice bearing MDA-MB-231 xenografts (bottom), *p<0.05 and ** p<0.01, and (D) ARN3261 and paclitaxel showed synergistic cytotoxicity judging by CI value less than 1.

### DETAILED DESCRIPTION

### Overview

Provided are methods of increasing the duration of remission for cancer comprising administering to a patient in need thereof a poly (ADP-ribose) polymerase (PARP) inhibitor and a salt-induced kinase 2 (SIK2) inhibitor. Also provided are methods of enhancing the sensitivity of PARP inhibitors for cancer, comprising administering to a patient in need thereof the poly (ADP-ribose) polymerase (PARP) inhibitor together with a salt-induced kinase 2 (SIK2) inhibitor. Also provided are methods of reducing hematologic toxicity during cancer treatment comprising administering to a patient in need thereof a poly (ADP-ribose) polymerase (PARP) inhibitor and a salt-induced kinase 2 (SIK2) inhibitor. Also provided are methods of maintaining hematopoiesis during cancer treatment comprising administering to a patient in need thereof a poly (ADP-ribose) polymerase (PARP) inhibitor and a salt-induced kinase 2 (SIK2) inhibitor. Also provided are methods of reducing toxicity and maintaining hematopoiesis during cancer treatment comprising administering to a patient in need thereof a poly (ADP-ribose) polymerase (PARP) inhibitor and a salt-induced kinase 2 (SIK2) inhibitor. Also provided are methods of increasing the duration of remission for cancer and maintaining hematopoiesis comprising administering to a patient in need thereof a poly (ADP-ribose) polymerase (PARP) inhibitor and a salt-induced kinase 2 (SIK2) inhibitor. Also provided are methods of preventing hematologic side effects during cancer treatment comprising administering to a patient in need thereof a poly (ADP-ribose) polymerase (PARP) inhibitor and a salt-induced kinase 2 (SIK2) inhibitor.

### Poly (ADP-Ribose) Polymerase Inhibitors (PARPi) and Salt Induced Kinase 2 (SIK2) Inhibitors for Treatment of Cancer

Poly (ADP-ribose) polymerase inhibitors (PARPi) have had an increasing role in the treatment of ovarian and breast cancers. PARPi are selectively active in cells with homologous recombination deficiency (HRD) caused by mutations in BRCA1/2, and other DNA repair pathway genes. Cancers with homologous recombination sufficiency respond poorly to PARPi. Cancers that initially respond to PARPi eventually develop drug resistance. Identified and disclosed herein are Salt Induced Kinase 2 inhibitors (SIK2i), Compound A and Compound B, which decrease DNA double-strand break (DSB) repair functions and produce synthetic lethality with multiple PARPi's in both HRD and HR-competent cells. SIK2 is required for centrosome splitting and PI3K activation and regulates cancer cell proliferation, metastasis and sensitivity to paclitaxel. As described herein, SIK2i sensitize ovarian and triple-negative breast cancer (TNBC) cells and xenografts to PARPi. SIK2i inhibit PARP enzyme activity and phosphorylation of class-IIa histone deacetylase (HDAC) 4/5/7. Furthermore, SIK2i abolish class-IIa HDAC 4/5/7-associated transcriptional activity of MEF2D, decreasing MEF2D binding to regulatory regions with high-chromatin accessibility in FANCD2, EXO1, and XRCC4 genes, resulting in repression of their functions in the DNA DSB repair pathway. Combination of PARPi and SIK2i provides a novel therapeutic strategy to enhance PARPi sensitivity for ovarian and triple-negative breast cancers.

Salt induced kinase 2 (SIK2) is an AMP kinase-related protein kinase that is required for ovarian cancer cell proliferation and metastasis. The kinase phosphorylates multiple substrates including cNAP1, triggering centrosome splitting, and the regulatory subunit of PI3K, enhancing the pathway's activity. SIK2 also phosphorylates class-IIa HDACs and controls their nuclear/cytoplasm shuttling, thus influencing the activity and nuclear localization of class-IIa HDACs. SIK2 is overexpressed and correlates with poor prognosis in patients with high-grade serous ovarian carcinoma (HGSOC). As described herein, orally administered low molecular weight drugs (Compound A and Compound B) were developed that inhibit SIK2 at nM concentrations, inhibit growth of ovarian cancer cell lines with an IC₅₀ of 0.8 to 2.6 μM, and inhibit growth of ovarian cancer xenografts, enhancing sensitivity to chemotherapeutic drugs such as paclitaxel or carboplatin.

Compound A is 3-(3,5-difluoro-2-methoxyphenyl)-5-(1-(piperidin-4-yl)-1H-pyrazol-4-yl)-1H-pyrrolo[2,3-b]pyridine and the structure is as follows:

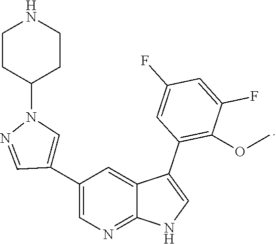 [SMILES: COc1c(F)cc(F)cc1-c1c[nH]c2ncc(-c3cnn(C4CCNCC4)c3)cc12]

Compound B is 3-(3,5-difluoro-2-methoxyphenyl)-5-(1-(1-(methylsulfonyl)piperidin-4-yl)-1H-pyrazol-4-yl)-1H-pyrrolo[2,3-b]pyridine and the structure is as follows:

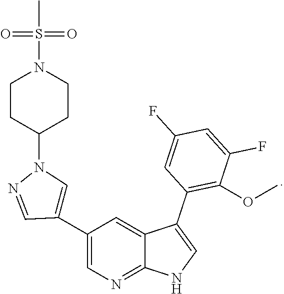 [SMILES: COc1c(F)cc(F)cc1-c1c[nH]c2ncc(-c3cnn(C4CCN(S(C)(=O)=O)CC4)c3)cc12]

Approximately half of HGSOC and TNBC exhibit aberrations in the homologous recombination (HR) and other DNA DSB repair pathways. HGSOC and TNBC with mutations of BRCA1 or BRCA2 are highly sensitive to PARPi. A fraction of HGSOC and TNBC without BRCA1/2 mutations have HR repair deficiency (HRD) and are also susceptible to PARPi. Three different PARPi (Olaparib, Rucaparib, and Niraparib) have been approved for use in ovarian cancer, and two different PARPi (Olaparib and Talazoparib) have been approved for use in breast cancer. Other PARPi include veliparib, iniparib, CEP9722, BMD-673, and E7016. Despite promising clinical results for PARPi as single agents, high dosage requirements and prevalence of acquired resistance remain challenges to more effective treatment. Combination therapies are of considerable interest for minimizing PARPi concentration and enhancing its efficiency. The present study discovered that SIK2 inhibitors enhance response to PARP inhibitors in ovarian and triple-negative breast cancer cell lines and xenografts independent of BRCA mutation status.

### Class-IIa Histone Deacetylases (HDACs)

Class-IIa histone deacetylases (HDACs) are involved in the regulation of multiple cellular responses. They generally act at the apex of specific genetic programs, by influencing the landscape of genes expressed in a specific context. Class-IIa HDACs do not bind directly to DNA, but rather interact with a selected number of transcription factors, such as Myocyte Enhancer Factor-2 (MEF2), that are recruited to specific genomic regions in a sequence-dependent manner MEF2 is a MADS box transcription factor originally discovered as a regulator of cardiogenesis and myogenesis. MEF2 influences the expression of numerous genes, individually and cooperatively with other transcription factors. MEF2 can also operate as a transcriptional repressor when complexed with class-IIa HDACs. However, the link between the repressor function of MEF2—class-IIa HDAC axis and expression of DNA repair genes in cancers is not well established.

### Methods of Treatment for Cancer

Provided herein are methods of increasing the magnitude and/or duration of activity of a poly (ADP-ribose) polymerase (PARP) inhibitor in a patient being treated with the PARP inhibitor, comprising administering to the patient in need thereof the PARP inhibitor and a salt-induced kinase 2 (SIK2) inhibitor.

Also provided are methods of increasing the sensitivity of cancer cells to treatment with a PARP inhibitor, comprising contacting the cells with the PARP inhibitor and a SIK2 inhibitor.

Also provided are methods of prolonging survival in a cancer patient in need thereof comprising administering to the patient in need thereof a PARP inhibitor and a SIK2 inhibitor.

Also provided are methods of suppressing tumor growth in a cancer patient in need thereof, comprising administering to the patient in need thereof a PARP inhibitor and a SIK2 inhibitor.

Also provided are methods of increasing the duration of remission for a cancer patient in need thereof comprising administering to the patient in need thereof a PARP inhibitor and a SIK2 inhibitor.

Also provided are methods of preventing a relapse or reducing the incidence of relapse of a cancer patient in remission, the method comprising administering to the patient in need thereof a PARP inhibitor and a SIK2 inhibitor.

Also provided are methods for reducing incidences of, or risk of, cancer recurrence in a patient deemed to be at risk of cancer recurrence, the method comprising administration to the subject of a PARP inhibitor and a SIK2 inhibitor.

Also provided herein are methods of reducing hematologic toxicity during cancer treatment comprising administering to a patient in need thereof a PARP inhibitor and a SIK2 inhibitor.

Also provided herein are methods of maintaining hematopoiesis during cancer treatment comprising administering to a patient in need thereof a PARP inhibitor and a SIK2 inhibitor.

Also provided herein are methods of reducing toxicity and maintaining hematopoiesis during cancer treatment comprising administering to a patient in need thereof a PARP inhibitor and a SIK2 inhibitor.

Also provided herein are methods of preventing hematologic side effects during cancer treatment comprising administering to a patient in need thereof a PARP inhibitor and a SIK2 inhibitor.

In some embodiments, the methods described herein may be used to treat cancer in a patient as described herein. In some embodiments, the type of cancer to be treated as described herein may be a cancer type that harbors one or more DNA repair deficiencies described herein. In some embodiments, a cancer type with one or more DNA repair deficiencies is sensitive to PARP inhibitors. In some embodiments, the type of cancer to be treated as described herein may include, but is not limited to, ovarian cancer, breast cancer, prostate cancer, and pancreatic cancer. In some embodiments, the type of cancer to be treated is ovarian cancer or breast cancer. Certain types of ovarian or breast cancer may be particularly suited for treatment as described herein, such as including, but not limited to, high-grade serous ovarian carcinoma (HGSOC) or triple-negative breast cancer. In some embodiments, the ovarian cancer is primary cancer. In some embodiments, the ovarian cancer is recurrent cancer. In some embodiments, the patient achieves remission for cancer and the cancer recurs. In some embodiments, the breast cancer is triple-negative breast cancer and has a mutation in BRCA1/2.

In some embodiments, the type of cancer to be treated as described herein is chosen from prostate cancer, pancreatic cancer, glioblastoma, melanoma, small cell lung cancer, non-small cell lung cancer, gastric cancer, fallopian tube cancer, peritoneal cancer, and testicular cancer.

In some embodiments, the type of cancer to be treated is prostate cancer. In some embodiments, the type of cancer to be treated is pancreatic cancer. In some embodiments, the type of cancer to be treated is glioblastoma. In some embodiments, the type of cancer to be treated is melanoma. In some embodiments, the type of cancer to be treated is small cell lung cancer (SCLC). In some embodiments, the type of cancer to be treated is non-small cell lung cancer. In some embodiments, the type of cancer to be treated as described herein is gastric cancer. In some embodiments, the type of cancer to be treated as described herein is fallopian tube cancer. In some embodiments, the type of cancer to be treated as described herein is peritoneal cancer. In some embodiments, the type of cancer to be treated as described herein is testicular cancer.

In some embodiments, the type of cancer to be treated is a tumor with compromised homologous recombination (HR)-mediated DNA repair.

In some embodiments, the type of cancer to be treated as described herein is a BRCA1/2-mutant solid tumor.

In some embodiments, the type of cancer to be treated as described herein is a BRCA-independent tumor with compromised HR-mediated DNA repair.

In some embodiments, the treatment occurs outside of a clinical trial setting.

In some embodiments, a PARP inhibitor and a SIK2 inhibitor as described herein may be administered in a clinical setting or may be administered in an alternate setting as deemed appropriate by a clinician or practitioner.

In some embodiments, administration of the SIK2 inhibitor to a patient who has already received, or is receiving, a PARP inhibitor, inhibits growth of ovarian or breast cancer cells in the primary or recurrent cancer. Administration of the SIK2 inhibitor in combination with the PARP inhibitor results in inhibition of growth of the cancer cells, or a reduction of tumor volume or size, or a reduction of symptoms associated with cancer. In some embodiments, administration of a SIK2 inhibitor in combination with a PARP inhibitor results in a reduction of side effects associated with cancer treatment, for example hematologic side effects, such as a reduction of white blood cells or red blood cells. Reduction of or decrease in white blood cells or red blood cells can also be associated with toxicity as a result of treatment with a PARP inhibitor, which can be ameliorated or reduced by the addition of a SIK2 inhibitor with the PARP inhibitor as described herein.

In some embodiments, a PARP inhibitor and a SIK2 inhibitor as described herein may be combined with other therapies or treatments for cancer in a patient. Other drug treatments that may be used to treat cancer in combination with a PARP inhibitor and a SIK2 inhibitor as described herein may include any chemotherapeutic drug and/or any immunotherapy drug known or available in the art. Any such drug treatments may be used as deemed appropriate by a clinician. A drug treatment that may be administered to a patient in combination with a PARP inhibitor and a SIK2 inhibitor for treatment of cancer as described herein may include, but is not limited to, Evista (Raloxifene Hydrochloride), Raloxifene Hydrochloride, Soltamox (Tamoxifen Citrate), Tamoxifen Citrate, Abemaciclib, Abraxane (Paclitaxel Albumin-stabilized Nanoparticle Formulation), Ado-Trastuzumab Emtansine, Afinitor (Everolimus), Afinitor Disperz (Everolimus), Alkeran (Melphan), Alpelisib, Anastrozole, Aredia (Pamidronate Disodium), Arimidex (Anastrozole), Aromasin (Exemestane), Atezolizumab, Avastin (Bevacizumab), Bevacizumab, Capecitabine, Carboplatin, Cisplatin, Cyclophosphamide, Docetaxel, Doxorubicin Hydrochloride, Doxil (Doxorubicin Hydrochloride Liposome), Ellence (Epirubicin Hydrochloride), Enhertu (Fam-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Femara, (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Hycamtin (Topotecan Hydrochloride), Ibrance (Palbociclib), Infugem (Gemcitabine Hydrochloride), Ixabepilone, Ixempra (Ixabepilone), Kadcyla (Ado-Trastuzumab Emtansine), Keytruda (Pembrolizumab), Kisqali (Ribociclib), Lapatinib Ditosylate, Letrozole, Lynparza (Olaparib), Margenza (Margetuximab-cmkb), Margetuximab-cmkb, Megestrol Acetate, Melphalan, Methotrexate Sodium, Neratinib Maleate, Nerlynx (Neratinib Maleate), Niraparib Tosylate Monohydrate, Olaparib, Paclitaxel, Paclitaxel Albumin-stabilized Nanoparticle Formulation, Palbociclib, Pamidronate Disodium, Pembrolizumab, Perjeta (Pertuzumab), Pertuzumab, Pertuzumab, Rubraca (Rucaparib Camsylate), Trastuzumab, and Hyaluronidase-zzxf, Phesgo (Pertuzumab, Trastuzumab, and Hyaluronidase-zzxf), Piqray (Alpelisib), Ribociclib, Sacituzumab Govitecan-hziy, Soltamox (Tamoxifen Citrate), Talazoparib Tosylate, Talzenna (Talazoparib Tosylate),Tamoxifen Citrate, Taxol, Taxotere (Docetaxel), Tecentriq (Atezolizumab), Tepadina (Thiotepa), Thiotepa, Topotecan Hydrochloride, Toremifene, Trastuzumab, Trastuzumab and Hyaluronidase-oysk, Trexall (Methotrexate Sodium), Trodelvy (Sacituzumab Govitecan-hziy), Tucatinib, Tukysa (Tucatinib), Tykerb (Lapatinib Ditosylate), Verzenio (Abemaciclib), Vinblastine Sulfate, Xeloda (Capecitabine), Zejula (Niraparib Tosylate Monohydrate), Zoladex (Goserelin Acetate). Any other drugs known or available in the art may also be used in combination with a PARP inhibitor and a SIK2 inhibitor as described herein without deviating from the scope of the present disclosure.

In some embodiments, a patient may be treated for ovarian cancer with a PARP inhibitor as described herein. Useful PARP inhibitors for treatment of ovarian cancer may include, but are not limited to, Olaparib, Rucaparib, and Niraparib. In some embodiments, a patient may be treated for breast cancer with a PARP inhibitor as described herein. Useful PARP inhibitors for treatment of breast cancer may include, but are not limited to, Olaparib and Talazoparib.

In some embodiments, a SIK2 inhibitor described herein may be used to treat ovarian cancer in combination with a PARP inhibitor described herein. Useful SIK2 inhibitors for treatment of ovarian or breast cancer as described herein include, but are not limited to, Compound A or Compound B. In some embodiments, the SIK2 inhibitor is Compound A. In some embodiments, the SIK2 inhibitor is Compound B.

As would be understood by one of skill in the art, a PARP inhibitor and a SIK2 inhibitor as described herein are administered in any form necessary or useful to the subject for treatment of cancer, for example, a liquid (e.g., injectable and infusible solutions), a semi-solid, a solid, an aqueous solution, a suspension, an emulsion, a gel, a magma, a mixture, a tincture, a powder, a capsule, a dispersion, a tablet, a pellet, a pill, a powder, a liposome, a lozenge, a troche, a liniment, an ointment, a lotion, a paste, a suppository, a spray, an inhalant, or the like. In some embodiments, a drug as described herein for treatment of cancer may be administered in a liquid or aqueous form for injection into a patient. The form can depend on the intended mode of administration and therapeutic application. Typically, compositions for the agents described herein are in the form of injectable or infusible solutions.

In some embodiments, a drug as described herein for treatment of cancer in a patient may be administered by any route or mode of administration, such as intravenous, oral, sublingual, rectal, vaginal, ocular, otic, nasal, cutaneous, enteral, epidural, intra-arterial, intravascular, nasal, respiratory, subcutaneous, topical, transdermal, intramuscular, or the like. In some embodiments, a PARP inhibitor and a SIK2 inhibitor as described herein are both administered orally (PO).

In some embodiments, a SIK2 inhibitor described herein, such as Compound A or Compound B, blocks DNA double-strand break (DSB) repair in the cancer cells, preventing the cancer cells from repairing damage and thereby resulting in apoptosis (i.e., death) of the cancer cell. Blocking DSB repair by a SIK2 inhibitor as described herein increases nuclear localization of histone deacetylase (HDAC) 4/5, which increases nuclear localization of HDAC4/5 and blocks the activity of transcription factors associated with DNA DSB repair. In some embodiments, the transcription factor associated with DNA DSB repair as described herein is a myocyte enhancer factor-2 (MEF2) protein, such as MEF2D. Thus, in some embodiments, the combination of a PARP inhibitor and a SIK2 inhibitor as described herein induces increased levels of apoptosis in the breast or ovarian cancer cells compared to cancer cells treated with only a PARP inhibitor or a SIK2 inhibitor.

In some embodiments, the combination of a PARP inhibitor and a SIK2 inhibitor as described herein enhances the sensitivity of the breast or ovarian cancer cells to a chemotherapeutic or immunogenic drug described herein, such as paclitaxel. Therefore, the administration of the PARP inhibitor and the SIK2 inhibitor as described herein enhances the activity of other cancer treatment drugs. For example, in some embodiments, the addition of a SIK2 inhibitor to treat ovarian or breast cancer being treated with a PARP inhibitor (e.g., Olaparib) enhances the inhibition of the PARP enzyme in the patient by the PARP inhibitor to produce a synergistic growth inhibition of the cancer cells. In some embodiments, the combination of the PARP inhibitor and the SIK2 inhibitor decreases expression of one or more genes involved in regulation of DNA repair and apoptosis in the cancer cell compared to cells treated with a PARP inhibitor (e.g., Olaparib) alone. Any gene involved in regulation of DNA repair and apoptosis can be inhibited with a PARP inhibitor and a SIK2 inhibitor as described herein, for example, one or more of BRCA2, EXO1, FANCD2, LIG4, XRCC4, BAX, BCL2, CASP7, and TRADD. Such genes are decreased or down-regulated by decreasing or eliminating the binding of a transcription factor (e.g., MEF2D) to the promoter regions of the genes, thereby decreasing expression of these genes, which results in the inability of the cancer cells to repair DNA, leading to apoptosis. In some embodiments, the combination of a PARP inhibitor and a SIK2 inhibitor may decrease the expression or activity of EXO1, FANCD2, and XRCC4, which result in death of the ovarian or breast cancer cells as described herein.

Unless otherwise specified herein, the methods described herein can be performed in accordance with the procedures exemplified herein or routinely practiced methods well known in the art. The following sections provide additional guidance for practicing the methods of the present disclosure.

### Pharmaceutical Compositions

In some embodiments, a PARP inhibitor and a SIK2 inhibitor may be administered together as a single composition, i.e., both drugs may be combined together in a solution or other drug form as described herein. In some embodiments, each drug may be administered separately (while still being administered concurrently), i.e., in separate solutions or drug forms as described herein. For example, a PARP inhibitor as described herein may be administered to a patient in an aqueous solution for intravenous administration, and a SIK2 inhibitor may be administered in a separate or distinct aqueous solution for intravenous administration. Pharmaceutical formulation is well established and known in the art.

In some embodiments, a PARP inhibitor and a SIK2 inhibitor may be formulated with excipient materials, such as sodium citrate, sodium dibasic phosphate heptahydrate, sodium monobasic phosphate, Tween-80, and a stabilizer. The PARP inhibitor and the SIK2 inhibitor can be provided, for example, in a buffered solution at a suitable concentration and can be stored at an appropriate temperature to maintain the efficacy of the drug(s), for example a temperature of 2-8° C. In some other embodiments, the pH of the composition is between about 5.5 and about 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5).

A pharmaceutical composition described herein can also include agents that reduce aggregation of the drug when formulated. Examples of aggregation reducing agents include one or more amino acids selected from the group consisting of methionine, arginine, lysine, aspartic acid, glycine, and glutamic acid. The pharmaceutical compositions can also include a sugar (e.g., sucrose, trehalose, mannitol, sorbitol, or xylitol) and/or a tonicity modifier (e.g., sodium chloride, mannitol, or sorbitol) and/or a surfactant (e.g., polysorbate-20 or polysorbate-80).

As described above for PARP inhibitors and SIK2 inhibitors, compositions comprising these drugs can be administered by a parenteral mode (e.g., intravenous, subcutaneous, intraperitoneal, or intramuscular injection). In one embodiment, a composition comprising a PARP inhibitor and/or a SIK2 inhibitor is administered intravenously. The phrases “parenteral administration” and “administered parenterally” as used herein mean modes of administration other than enteral and topical administration, usually by injection, and include, without limitation, intravenous, intramuscular, intra-arterial, intrathecal, intracapsular, intraocular, intracardiac, intradermal, intraperitoneal, transtracheal, subcutaneous, subcuticular, intraarticular, subcapsular, subarachnoid, intraspinal, epidural and intrasternal injection, and infusion.

A composition comprising a PARP inhibitor and/or a SIK2 inhibitor can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, a compositions comprising a PARP inhibitor and/or a SIK2 inhibitor may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In some embodiments, a composition comprising a PARP inhibitor and/or a SIK2 inhibitor is formulated in sterile distilled water or phosphate buffered saline. The pH of the pharmaceutical formulation may be between about 5.5 and about 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5).

### Administration of a PARP Inhibitor and a SIK2 Inhibitor

A PARP inhibitor and a SIK2 inhibitor as described herein can be administered to a subject, e.g., a patient in need thereof, by a variety of methods. For many applications, the route of administration is one of: intravenous injection or infusion (IV), subcutaneous injection (SC), intraperitoneally (IP), or intramuscular injection. Other modes of parenteral administration can also be used. Examples of such modes include: intra-arterial, intrathecal, intracapsular, intraocular, intracardiac, intradermal, transtracheal, subcuticular, intra-articular, subcapsular, subarachnoid, intraspinal, and epidural and intrasternal injection.

The route and/or mode of administration of the PARP inhibitor and the SIK2 inhibitor, or compositions comprising these, can also be tailored for the individual case, e.g., by monitoring the patient.

The composition(s) comprising a PARP inhibitor and/or a SIK2 inhibitor can be administered as a fixed dose, or in a mg/kg dose. The dose can also be chosen to reduce or avoid production of antibodies against the PARP inhibitor and the SIK2 inhibitor. Dosage regimens are adjusted to provide the desired response, e.g., a therapeutic response or a combinatorial therapeutic effect. Generally, doses of the PARP inhibitor and the SIK2 inhibitor (and optionally an additional agent) can be used in order to provide a subject with the agent in bioavailable quantities.

A PARP inhibitor and a SIK2 inhibitor can be administered, e.g., at a periodic interval over a period of time (a course of treatment) sufficient to encompass at least 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 does, 20 doses, or more, e.g., once or twice daily, or about one to four times per week, or such as weekly, biweekly (every two weeks), every three weeks, monthly, e.g., for between about 1 to 12 weeks, such as between 2 to 8 weeks, such as between about 3 to 7 weeks, such as for about 4, 5, or 6 weeks, or every 5 weeks, or every 6 weeks, or any interval deemed appropriate by a clinician. Factors that may influence the dosage and timing required to effectively treat a subject, include, e.g., the stage or severity of the disease or disorder, formulation, route of delivery, previous treatments, the general health and/or age of the subject, and other diseases present. Moreover, treatment of a subject with a therapeutically effective amount of a PARP inhibitor and a SIK2 inhibitor, or compositions comprising these, can include a single treatment or can include a series of treatments.

If a subject is at risk for developing a disorder described herein, the PARP inhibitor and the SIK2 inhibitor can be administered before the full onset of the disorder, e.g., as a preventative measure. The duration of such preventative treatment can be a single dosage of the composition or the treatment may continue (e.g., multiple dosages). For example, a subject at risk for the disorder or who has a predisposition for the disorder may be treated with a composition as described herein for days, weeks, months, or even years so as to prevent the disorder from occurring or fulminating.

For patients receiving treatment for ovarian or breast cancer, resistance of the cancer cells to the PARP inhibitor can reduce the efficacy of the drug. For these patients, administration of a combination of a PARP inhibitor and a SIK2 inhibitor can increase the sensitivity of cancer cells to the PARP inhibitor, thus prolonging the effects of the PARP inhibitor and thereby prolonging the survival of the patient having cancer.

In some embodiments, a PARP inhibitor and a SIK2 inhibitor may be administered to a patient in order to extend the duration of remission or to prevent a relapse or reduce the incidence of relapse of a cancer patient in remission. In other embodiments, a PARP inhibitor and a SIK2 inhibitor may be administered to a patient currently or previously receiving treatment for cancer with a PARP inhibitor in order to increase the magnitude and/or duration of activity of the PARP inhibitor in treating cancer in the patient.

In some embodiments, treatment of cancer in a patient using a drug treatment such as a PARP inhibitor or other drug described herein produces side effects in the patient, such as hematologic toxicity (i.e., a reduction or decrease in the numbers of red blood cells and/or white blood cells). Maintaining hematopoiesis in these patients (e.g., preventing a reduction in white or red blood cell numbers) can be beneficial and thus, administration of a combination of a PARP inhibitor and a SIK2 inhibitor to the patient reduces hematologic toxicity during cancer treatment.

A combination of a PARP inhibitor and a SIK2 inhibitor can be administered to a patient in need thereof (e.g., a patient that has had or is at risk of having breast or ovarian cancer) alone or in combination with (i.e., by co-administration or sequential administration) other therapeutic treatments or drugs for treating cancer (e.g., chemotherapeutic or immunotherapy drugs or treatments). In one embodiment, the additional therapeutic treatments or drugs are included in a pharmaceutical composition as described herein. In other embodiments, the additional therapeutic treatments or drugs are co-administered, administered concurrently, or administered sequentially in separate or distinct compositions.

### Kits

A PARP inhibitor and a SIK2 inhibitor for treatment of breast or ovarian cancer in a patient can be provided in a kit. In one embodiment, the kit includes (a) a container that contains the PARP inhibitor and the SIK2 inhibitor as described herein, and optionally (b) informational material. The informational material can be descriptive, instructional, marketing or other material that relates to the methods described herein and/or the use of the agents for therapeutic benefit.

In one embodiment, the kit also includes a second agent (e.g., a chemotherapeutic or immunotherapy drug described herein) for treating cancer described herein. For example, the kit includes a first container that contains the PARP inhibitor and the SIK2 inhibitor, and a second container that includes the chemotherapeutic or immunotherapy drug. In another embodiment, the kit includes a first container that contains the PARP inhibitor, a second container that contains the SIK2 inhibitor, and a third container that contains the chemotherapeutic or immunotherapy agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the PARP inhibitor and SIK2 inhibitor, as well as the chemotherapeutic or immunotherapy drug, e.g., in a suitable dose, dosage form, or mode of administration (e.g., a dose, dosage form, or mode of administration described herein), to treat a subject who has had or who is at risk for breast or ovarian cancer. The information can be provided in a variety of formats, include printed text, computer readable material, video recording, or audio recording, or information that provides a link or address to substantive material, e.g., on the internet.

In addition to the PARP inhibitor and SIK2 inhibitor, and including the chemotherapeutic or immunotherapy drug if applicable, the kit can include other ingredients, such as a solvent or buffer, a stabilizer, or a preservative. The PARP inhibitor and SIK2 inhibitor, and chemotherapeutic or immunotherapy drug, can be provided in any form described herein, e.g., liquid, dried or lyophilized form, substantially pure and/or sterile. When the agents are provided in a liquid solution, the liquid solution is an aqueous solution. When the agents are provided as a lyophilized product, the lyophilized powder is generally reconstituted by the addition of a suitable solvent. The solvent, e.g., sterile water or buffer (e.g., PBS), can optionally be provided in the kit.

The kit can include one or more containers for the drugs or compositions. In some embodiments, the kit contains separate containers, dividers or compartments for the drugs and informational material. For example, the PARP inhibitor and SIK2 inhibitor, and chemotherapeutic or immunotherapy drug, if applicable, can be contained in a bottle, vial, or syringe, and the informational material can be contained in a plastic sleeve or packet. In other embodiments, the separate elements of the kit are contained within a single, undivided container. For example, the PARP inhibitor and SIK2 inhibitor, and chemotherapeutic or immunotherapy drug, if applicable, is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the PARP inhibitor and SIK2 inhibitor, and chemotherapeutic or immunotherapy drug, if applicable, e.g., in a desired ratio. For example, the kit includes a plurality of syringes, ampules, foil packets, blister packs, or medical devices, e.g., each containing a single combination unit dose. The containers of the kits can be air-tight, waterproof (e.g., impermeable to changes in moisture or evaporation), and/or light-tight.

The kit optionally includes a device suitable for administration of the PARP inhibitor and SIK2 inhibitor, and chemotherapeutic or immunotherapy drug, if applicable, e.g., a syringe or other suitable delivery device. The device can be provided pre-loaded with one or both of the agents or can be empty, but suitable for loading.

### Definitions

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Unless defined otherwise, all technical and scientific terms used herein have the meaning commonly understood by one of ordinary skill in the art to which the disclosure pertains. Specific terminology of particular importance to the description of the present disclosure is defined below.

As used in this specification and the appended claims, the singular forms “a,” “an,” and “the,” along with similar references used in the context of describing a particular embodiment (especially in the context of certain of the following claims), can be construed to cover both the singular and the plural, unless specifically noted otherwise. Thus, for example, “an active agent” refers not only to a single active agent, but also to a combination of two or more different active agents, “a dosage form” refers to a combination of dosage forms, as well as to a single dosage form, and the like. In some embodiments, the term “or” as used herein, including the claims, is used to mean “and/or” unless explicitly indicated to refer to alternatives only or the alternatives are mutually exclusive.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. In some embodiments, “about” refers to a specified value +/−10%.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

As used herein, “co-administration” refers to the simultaneous administration of one or more drugs with another. In some embodiments, both drugs are administered at the same time. Co-administration may also refer to any particular time period of administration of either drug, or both drugs. For example, as described herein, a drug may be administered hours or days before administration of another drug and still be considered to have been co-administered. In some embodiments, co-administration may refer to any time of administration of either drug such that both drugs are present in the body of a patient at the same. In some embodiments, either drug may be administered before or after the other, so long as they are both present within the patient for a sufficient amount of time that the patient received the intended clinical or pharmacological benefits.

Conservative amino acid substitutions providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); Arginine (R), Lysine (K); 5) Isoleucine (I), Leucine (L), Methionine (M), Valine (V); and 6) Phenylalanine (F), Tyrosine (Y), Tryptophan (W). Not all residue positions within a protein will tolerate an otherwise “conservative” substitution. For instance, if an amino acid residue is essential for a function of the protein, even an otherwise conservative substitution may disrupt that activity, for example the specific binding of an antibody to a target epitope may be disrupted by a conservative mutation in the target epitope.

In some embodiments, conservative amino acid substitutions, e.g., substituting one acidic or basic amino acid for another, can often be made without affecting the biological activity of a recombinant polypeptide as described herein, Minor variations in sequence of this nature may be made in any of the peptides disclosed herein, provided that these changes do not substantially alter (e.g., by 15% or more) the desired activity of the protein,

As used herein, a dosage unit form or “fixed dose” as used herein refers to physically discrete units suited as unitary dosages for the patients to be treated; each unit contains a predetermined quantity of a PARP inhibitor and/or a SIK2 inhibitor calculated to produce the desired therapeutic effect in association with the required pharmaceutical carrier and optionally in association with the other agent. Single or multiple dosages may be given. Alternatively, or in addition, the PARP inhibitor and the SIK2 inhibitor, or composition(s) comprising these may be administered via continuous infusion.

A pharmaceutical composition(s) comprising a PARP inhibitor and/or a SIK2 inhibitor as described herein may include a “therapeutically effective amount” of the PARP inhibitor and/or a SIK2 inhibitor as described herein. The term “therapeutically effective amount,” “pharmacologically effective dose,” “pharmacologically effective amount,” or simply “effective amount” may be used interchangeably and refers to that amount of an agent effective to produce the intended pharmacological, therapeutic or preventive result, e.g., a reduction of cancerous cells or lessened cancer cell burden (i.e., reduction in number of cancer cells), tumor size, tumor density, lymph node involvement, metastases, or associated symptoms in the patient. The pharmacologically effective amount results in the amelioration of one or more symptoms of a disorder (e.g., ovarian or breast cancer), or prevents the advancement of a disorder, or causes the regression of the disorder, or prevents the disorder. Such effective amounts can be determined based on the effect of the administered agent, or the combinatorial effect of agents if more than one agent is used. A therapeutically effective amount of an agent may also vary according to factors such as the disease stage, state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects. In some examples, an “effective amount” is one that treats (including prophylaxis) one or more symptoms and/or underlying causes of cancer. In one example, an effective amount is a therapeutically effective amount. In one example, an effective amount is an amount that prevents one or more signs or symptoms of a particular disease or condition from developing.

As used herein, “gene expression” or “expression” refers to the process of gene transcription, translation, and post-translational modification.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt.

As used herein, “reducing” refers to a lowering or lessening, such as reducing cancer cell burden. In some embodiments, administration of a PARP inhibitor and a SIK2 inhibitor as described herein may result in “reduced” or lessened cancer cell burden (i.e., reduction in number of cancer cells), tumor size, tumor density, lymph node involvement, metastases, or associated symptoms in the patient compared to a patient not been administered such drugs. “Reducing” may also refer to a reduction in disease symptoms as a result of a treatment as described herein, either alone, or co-administered with another drug.

As used herein, “subject” or “individual” or “patient” refers to any patient for whom or which therapy is desired, and generally refers to the recipient of the therapy. A “subject” or “patient” refers to any animal classified as a mammal, e.g., human and non-human mammals. Examples of non-human animals include dogs, cats, cattle, horses, sheep, pigs, goats, rabbits, etc. Unless otherwise noted, the terms “patient” or “subject” are used herein interchangeably. In some embodiments, a subject amenable for therapeutic applications may be a primate, e.g., human and non-human primates.

The terms “treating” and “treatment” or “alleviating” as used herein refer to reduction or lessening in severity and/or frequency of symptoms, elimination of symptoms and/or underlying cause, and improvement or remediation of damage. In certain aspects, the term “treating” and “treatment” as used herein refer to the prevention of the occurrence of symptoms. In other aspects, the term “treating” and “treatment” as used herein refer to the prevention of the underlying cause of symptoms associated with a disease or condition, such as breast or ovarian cancer. The phrase “administering to a patient” refers to the process of introducing a composition or drug into the patient via an art-recognized means of introduction. “Treating” or “alleviating” also includes the administration of compounds or agents to a subject to prevent or delay the onset of the symptoms, complications, or biochemical indicia of a disease (e.g., breast or ovarian cancer), alleviating the symptoms or arresting or inhibiting further development of the disease, condition, or disorder. Subjects in need of treatment include those already suffering from the disease or condition, as well as those being at risk of developing the disease or condition. Treatment may be prophylactic (to prevent or delay the onset of the disease or condition, or to prevent the manifestation of clinical or subclinical symptoms thereof) or therapeutic suppression, or alleviation of symptoms after the manifestation of the disease or condition.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1—SIK2 Inhibition Sensitizes Ovarian and Breast Cancer Cells by Enhancing Olaparib-Mediated Inhibition of PARP Enzyme Activity

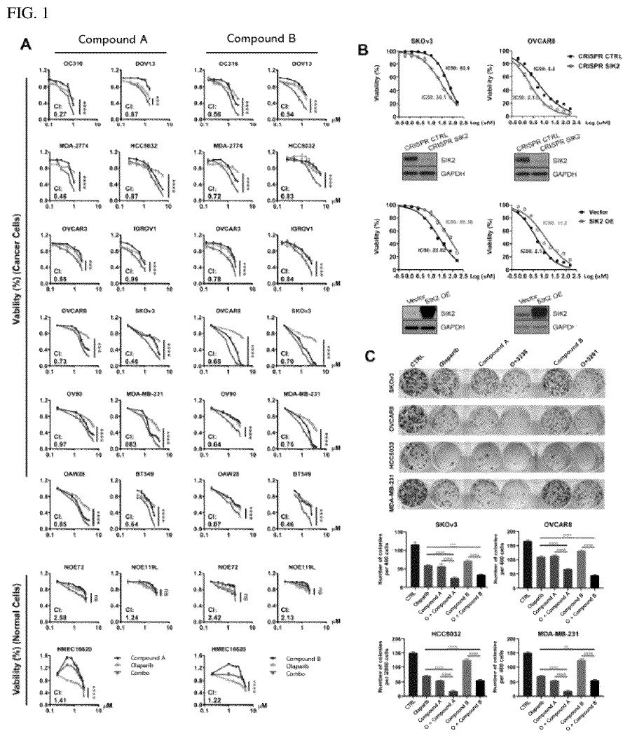

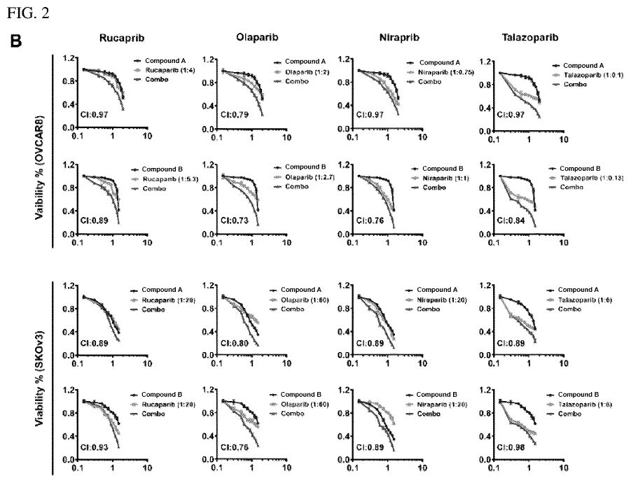

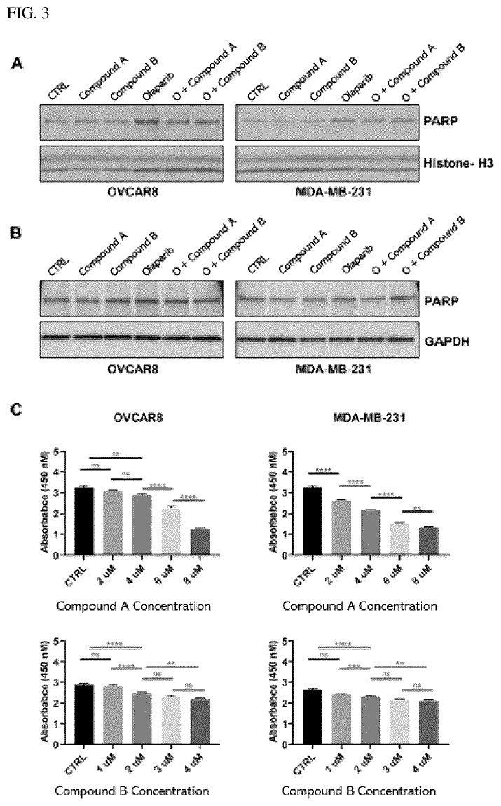

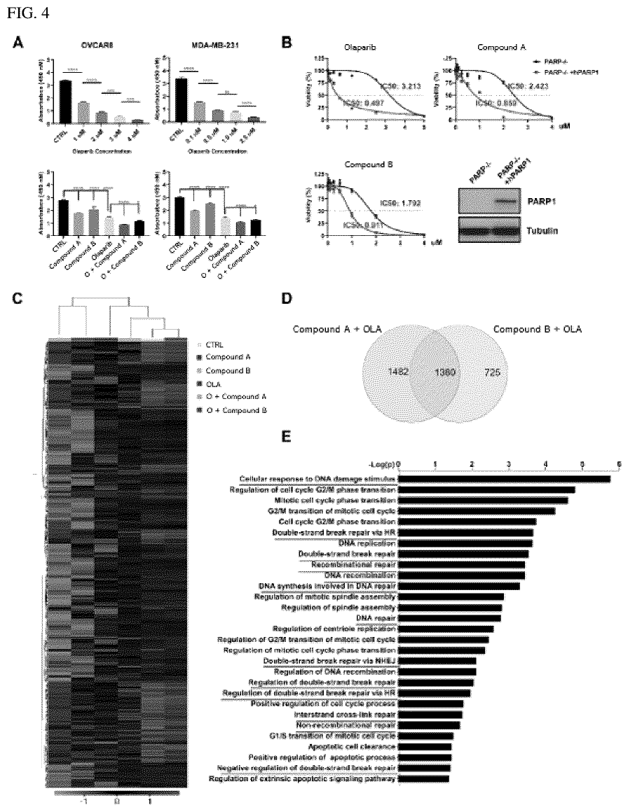

To explore whether modulation of SIK2 kinase activity can sensitize cancer cells to PARP inhibitors, the effect of combining a SIK2 kinase inhibitor (Compound A or Compound B) with olaparib was examined on cell growth in 10 ovarian and 2 triple-negative breast cancer cell lines as well as in normal cell lines (FIG. 1A). Sources and culture media for the cell lines described herein are provided in Table 1. Olaparib-induced growth inhibition (green line) was significantly enhanced by combination treatment (red line) with either Compound A or Compound B in all 12-cancer cell lines tested, but not in non-tumorigenic NOE72 and NOE119L (normal ovarian epithelial cells) and HMEC16620 (human mammary epithelial cells) (FIG. 1A). Moreover, all 12-cancer cell lines demonstrated synergistic growth inhibition with a combination of Compound A or Compound B with olaparib (combination index CI<1 using the CalcuSyn model), when compared to non-tumorigenic cells that did not undergo such a synergistic growth inhibition (FIG. 1A). To exclude potential off-target effects of SIK2 inhibitors, SIK2 was knocked down by CRISPR/Cas9 and stable ectopic expression of SIK2 was established in SKOv3 and OVCAR8 ovarian cancer cells. Knock-out of SIK2 sensitized cancer cells to olaparib judged by lower IC₅₀ (the concentration of a drug that gives half-maximal response, see Table 2) for olaparib in SIK2 deficient cells compared to control cells (FIG. 1B). In contrast, stable ectopic expression of SIK2 in SKOv3 and OVCAR8 cell lines desensitized cancer cells to olaparib, evidenced by an increased IC₅₀ of olaparib (FIG. 1B). Clonogenic assays were performed using three ovarian and one triple-negative breast cancer cell lines. Combination treatment with a SIK2 inhibitor and olaparib significantly decreased the number and size of colonies when compared to either the SIK2 inhibitor or olaparib alone (FIG. 1C and FIG. 2A). Furthermore, synergistic activity of SIK2 inhibition with PARP inhibition was evaluated with three structurally distinct PARP inhibitors (rucaparib, niraparib, and talazoparib) that have different PARP trapping potential. Although clinical PARP inhibitors can be ranked by their ability to trap PARP (from the most to the least potent): talazoparib>>niraparib>olaparib=rucaprib, SIK2 inhibitors synergized with PARP inhibitors with high (talazoparib) and low PARP trapping activity (olaparib) exhibiting similar combination indices (FIG. 2B). PARP binding in the chromatin fraction (indicative of PARP trapping) remained unchanged following treatment with SIK2 inhibitors, suggesting that SIK2 inhibitor-mediated enhancement of PARP inhibition is independent of PARP trapping activity (FIG. 3A). Measurement of PARP enzyme activity did, however, indicate that treatment with SIK2 inhibitors further decreased olaparib-induced suppression of PARP enzyme activity in cancer cells with detectable PARP protein levels (FIG. 4A and FIGS. 3B and 3C); consistent with the possibility that inhibition of PARP enzyme activity underlies the synergistic effect of SIK2 and PARP inhibition. To further test this possibility, DT40 PARP-1 −/−cells that lack PARP enzyme activity (avian cells lack PARP2) were treated with SIK2 inhibitors or olaparib. DT40 PARP-1 −/−cells resisted olaparib or SIK2 inhibitors, consistent with the lack of PARP1/2 (FIG. 4B). This is consistent with the synergistic effect of SIK2 inhibitors and olaparib depending upon the presence of PARP protein and PARP enzyme activity.

TABLE 1
Source and culture medium of cell lines.

| | | Culture | Tissue |
|---|---|---|---|
| Cell Line | Source | Medium | Classification |
| OC316 | Gordon Mills | RPMI 1640 | Ovarian Cancer |
| MDA-2774 | Gordon Mills | RPMI 1640 | Ovarian Cancer |
| OVCAR3 | Gordon Mills | RPMI 1640 | Ovarian Cancer |
| OVCAR8 | Gordon Mills | RPMI 1640 | Ovarian Cancer |
| OV90 | ATCC | 105 + 199 | Ovarian Cancer |
| OAW28 | ATCC | DMEM | Ovarian Cancer |
| DOV13 | Gordon Mills | DMEM | Ovarian Cancer |
| HCC5032 | Gordon Mills | RPMI 1640 | Ovarian Cancer |
| IGROV1 | Gordon Mills | RPMI 1640 | Ovarian Cancer |
| SKOv3 | ATCC | RPMI 1640 | Ovarian Cancer |
| MDA-MB-231 | ATCC | RPMI 1640 | Breast Cancer |
| BT549 | ATCC | RPMI 1640 | Breast Cancer |
| NOE72 | Robert Bast | 105 + 199 | Normal Ovarian |
| NOE119L | Robert Bast | 105 + 199 | Normal Ovarian |
| HMEC16620 | Robert Bast | 105 + 199 | Normal Breast |
| SKOv3 SIK2 KO | Ahmed Ahmed | RPMI 1640 | Ovarian Cancer |
| SKOv3 SIK2 KO | Ahmed Ahmed | RPMI 1640 | Ovarian Cancer |
| CTRL | | | |
| OVCAR8 SIK2 KO | Ahmed Ahmed | RPMI 1640 | Ovarian Cancer |
| OVCAR8 SIK2 KO | Ahmed Ahmed | RPMI 1640 | Ovarian Cancer |
| CTRL | | | |
| SKOv3 SIK2 OE | Ahmed Ahmed | RPMI 1640 | Ovarian Cancer |
| SKOv3 SIK2 OE | Ahmed Ahmed | RPMI 1640 | Ovarian Cancer |
| CTRL | | | |
| OVCAR8 SIK OE | Robert Bast | RPMI 1640 | Ovarian Cancer |
| OVCAR8 SIK2 OE | Robert Bast | RPMI 1640 | Ovarian Cancer |
| CTRL | | | |

TABLE 2
IC₅₀ values of inhibitors and concentration ration of SIK2 inhibitors to Olaparib.

| | Compound A | Compound B | Olaparib | Compound | Compound |
|---|---|---|---|---|---|
| Cell Line | (μM) (IC₅₀) | (μM) (IC₅₀) | (μM) (IC₅₀) | A:Olaparib | B:Olaparib |
| OVCAR8 | 1.396 | 1.765 | 6.396 | 1:0.5 | 1:0.5 |
| SKOv3 | 1.231 | 2.843 | 55.51 | 1:12.5 | 1:12.5 |
| HCC5032 | 2.243 | 2.397 | 14.52 | 1:3 | 1:1 |
| OC316 | 1.259 | 2.25 | 4.91 | 1:5 | 1:2 |
| IGROV1 | 1.084 | 1.605 | 2.83 | 1:5 | 1:2 |
| MDA-2774 | 1.081 | 0.592 | 6.289 | 1:5 | 1:2 |
| OV90 | 3.869 | 5.428 | 8.364 | 1:1 | 1:2 |
| DOV13 | 2.811 | 1.995 | 32.16 | 1:6 | 1:3 |
| OVCAR3 | 2.828 | 2.29 | 3.788 | 1:1 | 1:1 |
| OAW28 | 1.714 | 3.400 | 3.658 | 1:1 | 1:1.3 |
| MDA-MD- | 2.369 | 1.182 | 4.474 | 1:0.5 | 1:0.5 |
| 231 | | | | | |
| BT549 | 2.02 | 2.1 | 13.7 | 1:4 | 1:4 |
| NOE72 | 5.745 | 4.161 | 6.383 | 1:0.5 | 1:0.75 |
| NOE119L | 4.805 | 5.860 | 15.06 | 1:0.5 | 1:0.75 |
| HMEC16620 | 3.6 | 5.3 | 1.2 | 1:0.5 | 1:0.25 |

### Example 2—Compound A and Compound B Perturb Transcription of DNA Repair and Apoptosis Genes

While treatment of SIK2 inhibitors can enhance olaparib-mediated inhibition of PARP enzyme activity, it was asked whether SIK2 inhibitors might alter other key functional components of the DNA DSB repair pathways that might also contribute to the synergy observed between SIK2 and PARP inhibition. To explore this possibility, RNA-sequencing (RNA-seq) data was generated from perturbed SKOv3 cells and differential expression analysis was performed. The numbers of transcripts up or down-regulated by ≥2-fold after treatment with Compound A, Compound B, olaparib, Compound A+olaparib or Compound B+olaparib were 1308, 366, 3, 2862 and 2105, respectively. Based on a heatmap with unsupervised hierarchical clustering of 3587 transcripts altered by both Compound A+olaparib and Compound B+olaparib treatments (FIG. 4C), olaparib-treated and control groups shared relatively similar transcriptomes, whereas both SIK2 inhibitor and olaparib combination treatment groups clustered together. These data indicate that combination treatments showed the most significant alteration of transcripts compared to single agents alone and that SIK2 inhibition significantly induced olaparib-mediated transcriptional repression. (FIG. 4C). Using a Venn analysis, 1380 differentially expressed transcripts were shared by both SIK2 inhibitors and the olaparib combination treatment groups (FIG. 4D). Gene Ontology (GO) Biological Processes enrichment analysis of 1380 differentially expressed genes identified multiple aspects of regulation involving mitosis, DNA damage checkpoint, cell cycle, DNA repair and apoptosis (FIG. 4E), suggesting that SIK2 inhibition may enhance olaparib sensitivity by regulating DNA repair and apoptosis.

### Example 3—Compound A and Compound B Enhance Olaparib-Induced DNA DSB and Apoptosis

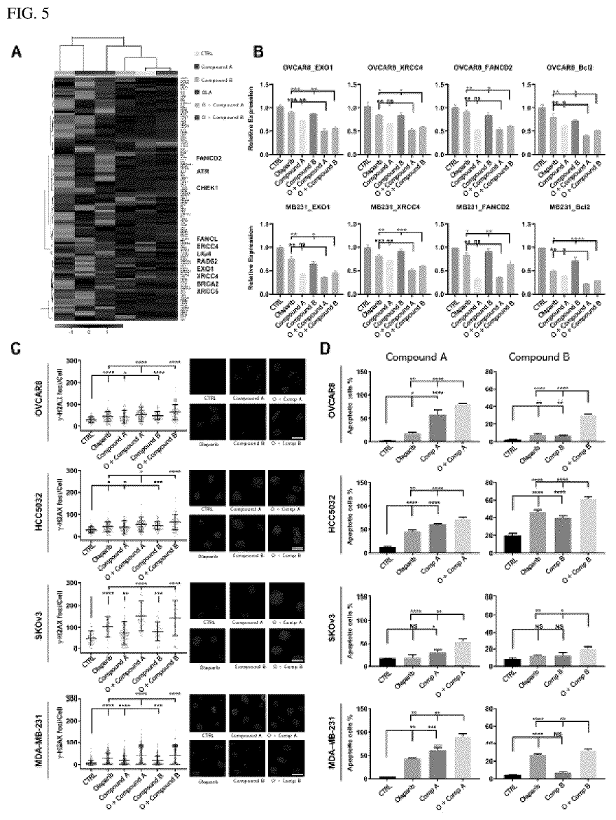

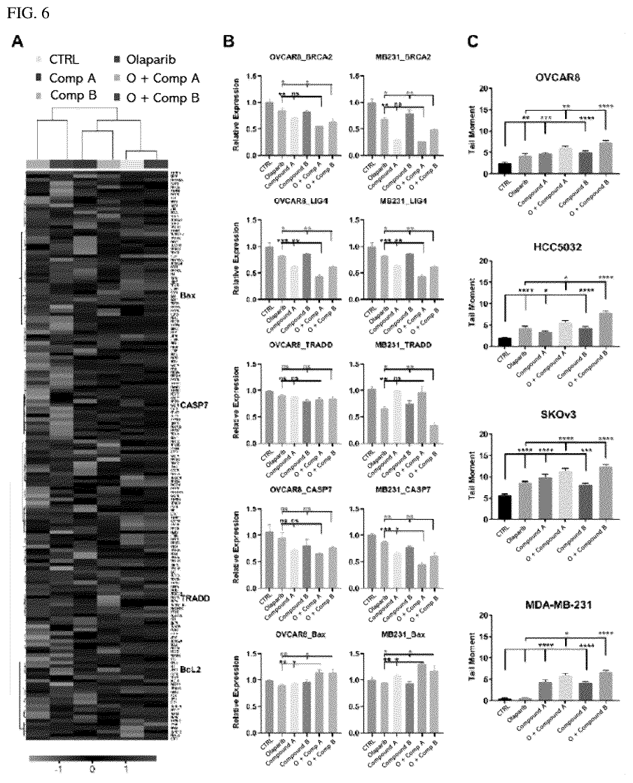

Detailed analysis of the expression of transcripts participating in regulation of DNA repair and apoptosis further demonstrated that SIK2 inhibition enhances PARP inhibition-mediated DNA repair (FIG. 5A) and apoptosis (FIG. 6A). To verify the RNA-seq results, nine genes involved in regulation of DNA repair and apoptosis (BRCA2, EXO1, FANCD2, LIG4, XRCC4, BAX, BCL2, CASP7, and TRADD) were selected and analyzed with RT-qPCR (quantitative reverse transcription PCR) using OVCAR8 ovarian cancer and MDA-MB-231 breast cancer cells. Treatment with Compound A or Compound B combined with olaparib (Compound A+olaparib or Compound B+olaparib) significantly decreased the expression of EXO1, XRCC4, FANCD2, BRCA2, LIG4, CASP7, and BCL2 and increased expression of BAX compared to olaparib treatment alone in both cell lines tested (FIG. 5B and FIG. 6B). Similar results were also observed in the cells treated with Compound B in combination with olaparib (FIG. 5B and FIG. 6B). These data are consistent with the observations documented in RNA-seq analysis.

To confirm whether SIK2 inhibitors induce DNA damage in cancer cells by inhibiting DNA repair, the effect of SIK2 inhibitors on olaparib-mediated induction of DNA DSBs was tested. Compound A, Compound B, or olaparib modestly increased levels of both phosphorylation of H2AX (γ-H2AX) and tailed DNA biomarkers, whereas combined treatment of SKOv3, OVCAR8, HCC5032, or MDA-MB-231 cells with Compound A or Compound B and olaparib increased the levels of γ-H2AX and the percentage of tailed DNA significantly (FIG. 5C and FIG. 6C), consistent with the possibility that SIK2 inhibition blocked DNA DSB repair. As unrepaired DSB can trigger apoptosis, Annexin V expression was measured to determine whether the combination of SIK2 inhibitor and olaparib induced greater levels of apoptosis. Compound A or Compound B combined with olaparib treatment induced significantly higher levels of apoptosis than did either single agent (FIG. 5D), consistent with the critical prerequisite of DNA DSB repair for cancer cell survival. Together, these results suggest that preventing DNA DSB repair by SIK2 inhibitors enhances the vulnerability of cancer cells to PARP inhibition.

### Example 4—SIK2 Inhibition Decreases Phosphorylation of Class IIa HDACs and Promoter Activity of MEF2 Transcription Factors

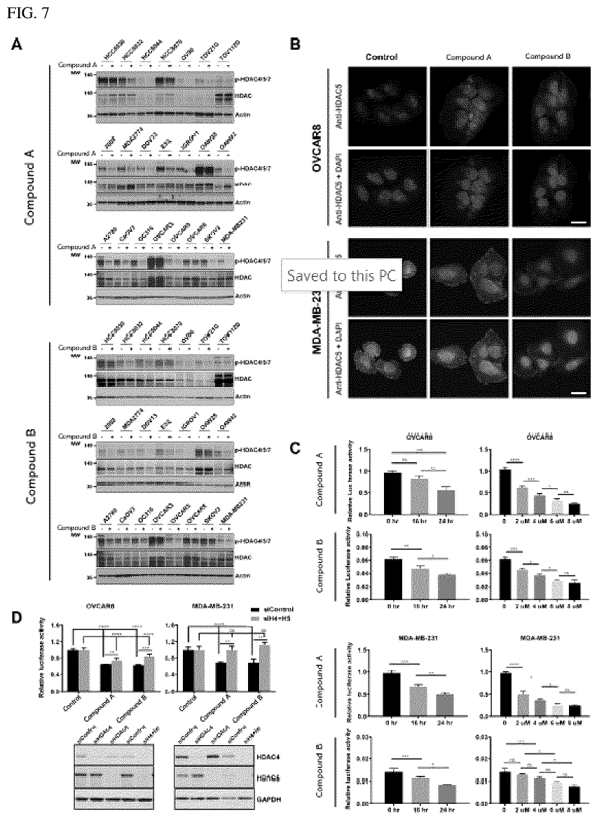

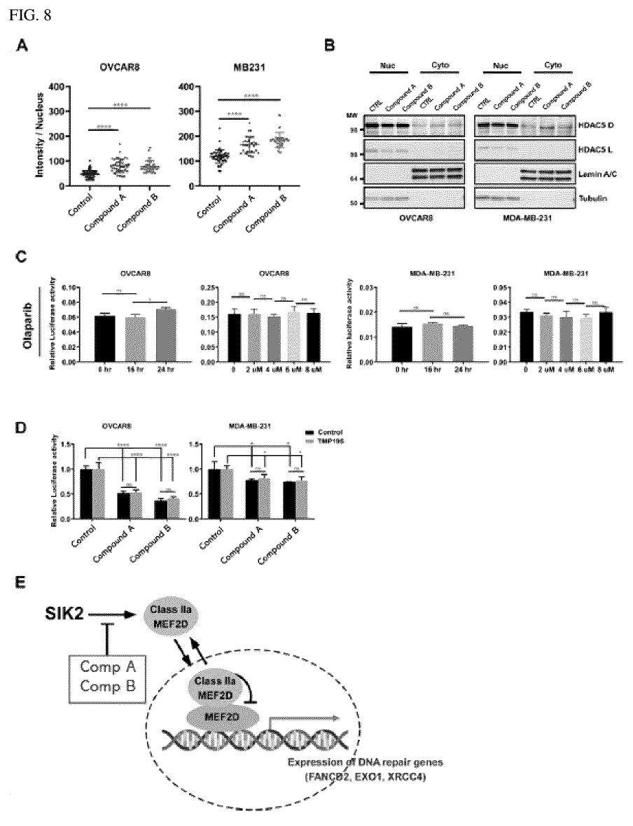

To identify the mechanism(s) by which SIK2 inhibition decreases DNA DBS repair, it was tested whether SIK2 inhibitors decrease the phosphorylation of class IIa HDACs, which control its nuclear cytoplasmic shuttling and consequently its association with DNA. Compound A or Compound B significantly decreased the phosphorylation of HDAC4 (Ser246)/HDAC5 (Ser256)/HDAC7 (Ser155) in nearly all the cell lines tested by western analysis using an antibody recognizing all three phosphorylation sites simultaneously (FIG. 4A). Next, it was investigated whether SIK2 inhibitors increase nuclear localization of HDAC5. SIK2 inhibition increased nuclear localization of HDAC5 judged by increasing nuclear florescence intensity (FIG. 7B and FIG. 8A) and the nuclear fraction of HDAC5 expression (FIG. 8B). This result raised the possibility that SIK2 inhibition downregulates expression of DNA repair genes by enhancing binding of HDAC5 with DNA-binding transcriptional factors, for which HDAC5 may serve as a transcriptional corepressor complex blocking the expression of MEF2 downstream targets. Therefore, it was hypothesized that SIK2 inhibition may block MEF transcription factor activity. To test this hypothesis, MEF2 promoter activity was measured using a luciferase reporter assay in ovarian and breast cancer cell lines, in the presence and absence of the SIK2 inhibitors Compound A, Compound B, or olaparib. SIK2 inhibitors significantly reduced MEF2 promoter activity in a time- and dose-dependent manner (FIG. 7C), but olaparib did not, as expected (FIG. 6C). Next, it was examined whether SIK2 regulation of MEF2 activity was HDAC4/5-dependent, increasing its binding to MEF2D protein. Knockdown of class IIa HDAC4/5 with siRNA prevented a Compound A or Compound B-mediated decrease of MEF2 promoter activity (FIG. 7D), but a decrease in MEF2 promoter activity was not prevented by inhibition of HDAC enzyme activity using TMP195, a selective class-IIa HDAC inhibitor (FIG. 8D). These observations are consistent with the hypothesis that SIK2 inhibition increases nuclear localization of HDAC4/5, blocking MEF2 transcription (FIG. 8E).

### Example 5—SIK2 Inhibition Alters MEF2D Transcription Factor-Mediated Downstream Signaling

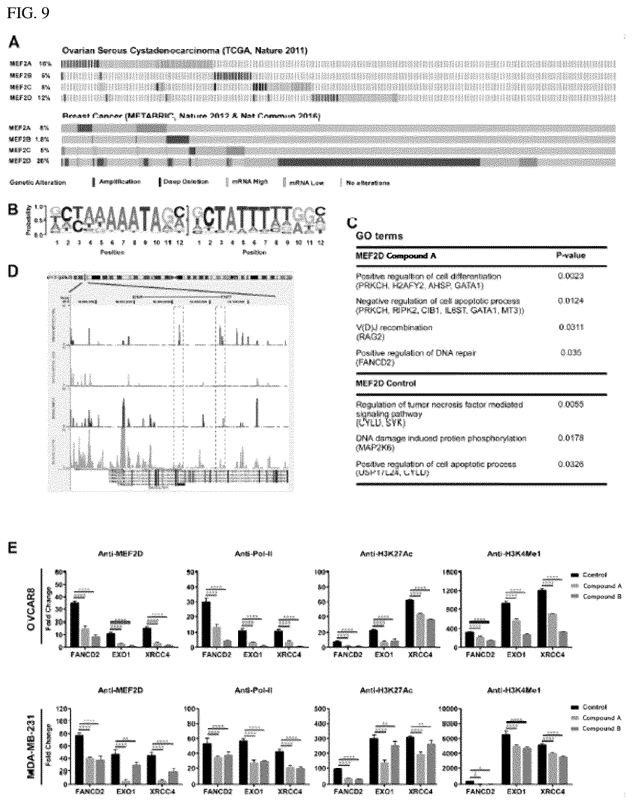

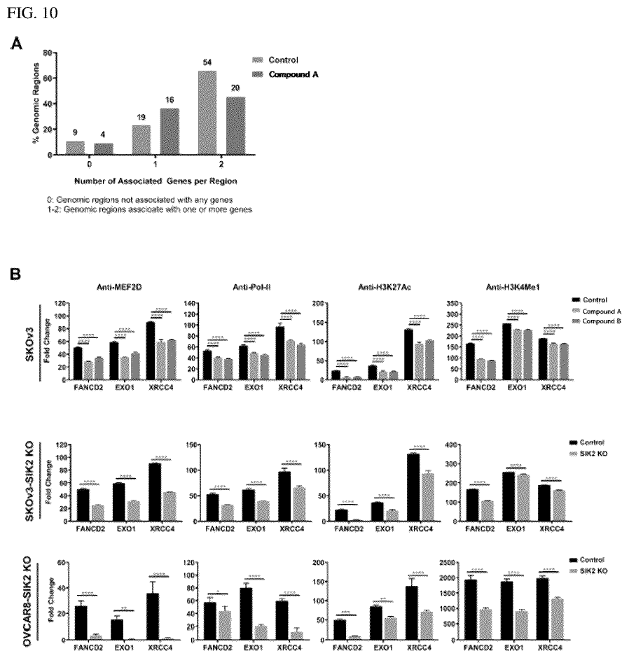

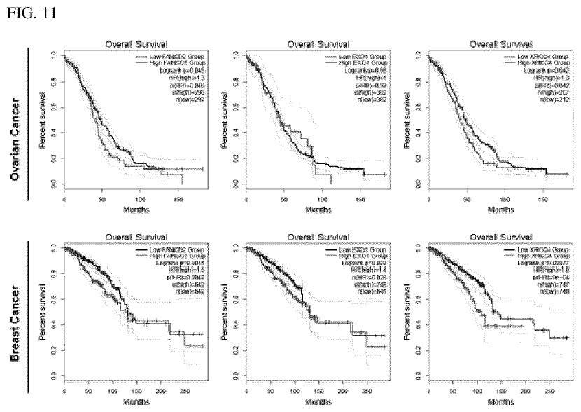

To explore the clinical relevance of the MEF2 transcription factors in ovarian and triple-negative breast cancers, alterations in the frequencies of individual MEF2 family members were examined in these tumor types. According to the cBioPortal TCGA database, 15-21% of ovarian and breast cancers contained amplification and mRNA upregulation of MEF2D (FIG. 9A). Genome-wide binding of MEF2D in SKOv3 ovarian cancer cells was then examined using chromatin immunoprecipitation sequencing (Chip-seq). In the genome-wide setting, 73 binding sites of MEF2D were identified and showed 50% reduction (36 binding sites) in the cells treated with Compound A (FIG. 10A). To identify a MEF2D consensus recognition sequence in ovarian cancer cells, de novo-motif discovery analysis was performed. A known MEF2 consensus recognition sequence could be detected in 59% (p=1e−9) of all random peaks analyzed (FIG. 9B). Moreover, motifs containing the consensus sequence for other TFs including Sox15, Usf2, and Sp1 were found at frequencies ranging from 19% to 34% suggesting that MEF2D can affect expression of downstream targets by associating with MEF2D DNA-binding site or by interacting with other transcription factors. This result is consistent with previous studies that have suggested MEF2D may function as a transcription factor or enhancer. In addition, GO enrichment analysis indicated that MEF2D-bound genes in control SKOv3 cells exhibited significant enrichment in positive regulation of cell differentiation, negative regulation of cell apoptotic processes, V(D) recombination and positive regulation of DNA repair. By contrast, several MEF2D-bound genes involved in regulation of the tumor necrosis factor mediated signaling pathway, DNA damage induced protein phosphorylation and positive regulation of cell apoptotic process were documented in cells treated with Compound A (FIG. 9C). Moreover, Chip-seq analysis indicated that MEF2D binds directly to FANCD2. FANCD2 plays a major role in homology-dependent repair (HDR)-mediated replication restart and in suppressing new origin firing. Chip-qPCR of FANCD2 confirmed MEF2D-association with the FANCD2 promoter/enhancer region. This association was decreased with SIK2 inhibition by Compound A or Compound B in all four cell lines assessed (FIG. 9E and FIG. 10B). Exonuclease I (EXO1) and X-ray repair cross-complementing protein 4 (XRCC4) are both downregulated by SIK2 inhibition in RNA-seq (FIG. 5A). EXO1 participates in extensive DSB end resection, an initial step in the homologous recombination (HR) pathway and XRCC4 is a component of the complex that mediates nonhomologous end-joining (NHEJ). Although EXO1 and XRCC4 genes were not associated with MEF2D peaks by Chip-seq analysis, which may due to poor quality of MEF2D antibody, the potential MEF2D binding sites at their promoter regions were identified. Chip-qPCR analysis revealed MEF2D binding to EXO1 and XRCC4 promoter/enhancer regions, and MEF2D binding affinities to those targets were significantly decreased with SIK2 inhibition by Compound A or Compound B in all cell lines tested (FIG. 9E and FIG. 10B). Notably, SIK2 inhibition also reduced H3K27Ac and H3K4Me1 RNA Pol-II at the FANCD2, EXO1, and XRCC4 promoter/enhancer regions (FIG. 9E and FIG. 10B). Both H3K27Ac and H3K4me1 are the activation marks of enhancers and have regulatory function to increase the transcription of target genes. PoI-II also is reported to regulate gene transcription by binding to both promoters and enhancers. Thus, these data support that FANCD2, EXO1, and XRCC4 are the direct targets of MEF2D and that SIK2 regulates DNA DSB repair by repression of MEF2D transcriptional activity. To evaluate the clinical relevance of the study, Kaplan-Meier survival analysis was examined, which showed that breast and ovarian patients with high expression of FANCD2 and XRCC4 have poorer overall survival than those with low expression of FANCD2 and XRCC4 (FIG. 11). EXO1 expression was also positively correlated with survival in breast cancer, but not in ovarian cancer (FIG. 11). These data are consistent with previous reports that overexpression of SIK2 correlates with poor prognosis in patients with ovarian and breast cancer.

### Example 6—Overexpression of MEF2D is Sufficient to Block SIK2 Inhibition-Induced DNA Damage and Growth Inhibition

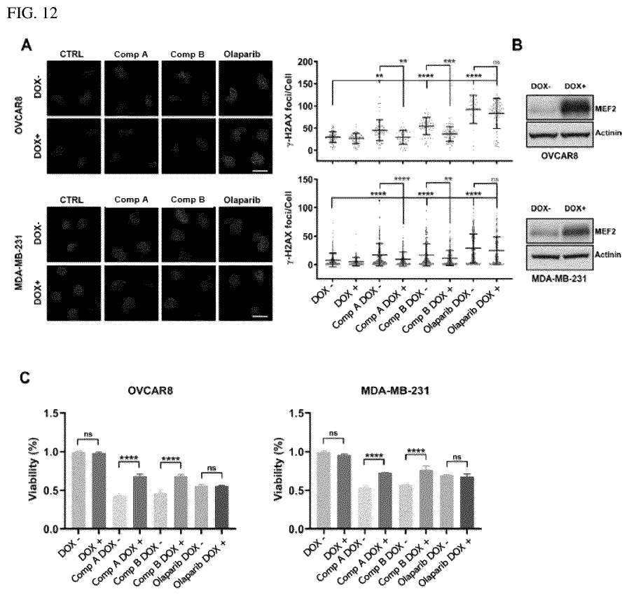

As describe above, SIK2 inhibition blocks HDAC4/MEF2-mediated DNA DSB repair by downregulating the expression of critical factors participating in this process. To test whether MEF2D downregulation was sufficient to explain the effects of SIK2 inhibition on DNA DSB repair and whether overexpression of MEF2D will rescue SIK2 inhibitor-mediated DNA damage and growth inhibition, OVCAR8 and MDA-MB-231 doxycycline (DOX)-inducible stable cell lines expressing MEF2D were generated. When MEF2D expression was induced by DOX treatment, γ-H2AX foci were significantly decreased in the cells treated with either Compound A (p<0.001 in MDA-MB-231 and p<0.0001 in OVCAR8 cells) or Compound B (p<0.0001 in both MDA-MB-231 and OVCAR8 cells), but not olaparib, compared to un-induced cells with no DOX treatment in both the OVCAR8 (p=0.4514) and MDA-MB-231 (p=0.3511) cell lines (FIGS. 12A and 12B). These data further confirm a role for MEF2D in promoting cancer survival by decreasing DNA damage in cancer cells. In addition, when viability was measured, induction of MEF2D partially rescued toxicity from Compound A or Compound B, but not from olaparib to cells with MEF2D induction (FIG. 12C). Together, these results suggest that SIK2 inhibitors enhance the vulnerability of cancer cells to olaparib not only by inhibiting PARP enzyme activity but also by blocking the class-IIa HDAC/MEF2D-mediated DNA repair function.

### Example 7—Co-Administration of SIK2 Inhibitor and Olaparib is Synergistic In Vivo

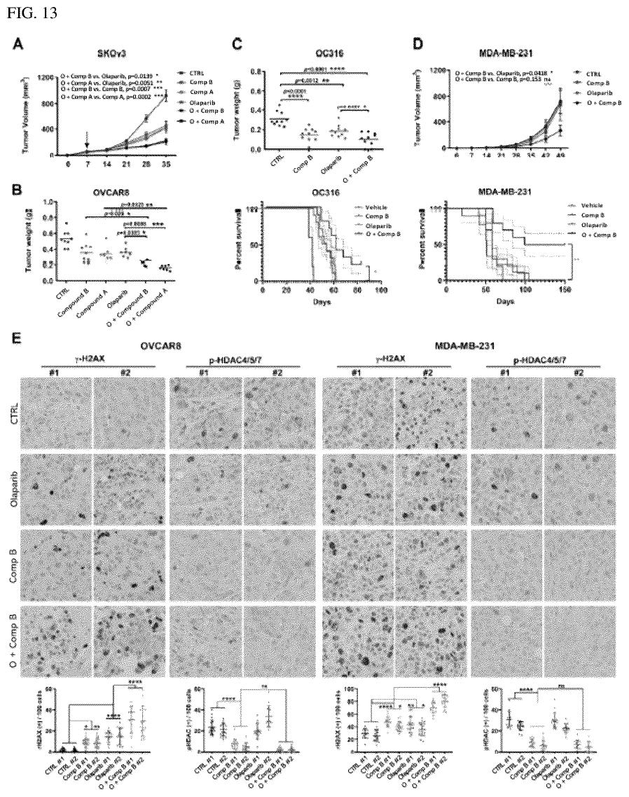

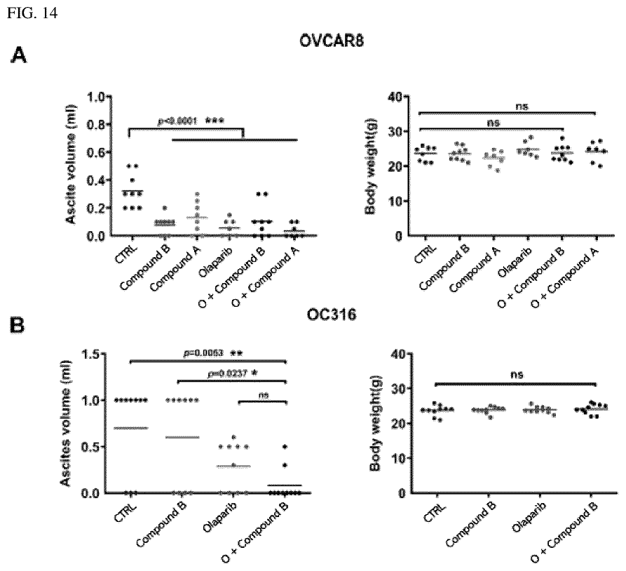

Based on enhancement of PARP inhibitor activity by SIK2 inhibition in cell culture, it was tested whether the addition of SIK2 inhibitors could promote PARP inhibitor response in vivo. When the BRCA-proficient SKOv3 cell line was injected subcutaneously into mice, treatment with Compound A, Compound B, or olaparib alone significantly inhibited tumor growth, compared to a vehicle control (FIG. 13A). The combination of Compound A+olaparib or Compound B+olaparib produced greater inhibition of tumor growth than either single agent (FIG. 13A). Another BRCA-proficient OVCAR8 ovarian cancer cell line was injected intraperitoneally into mice that were treated as described for the SKOv3 xenograft model. Compound A or Compound B in combination with olaparib combination significantly inhibited OVCAR8 tumor growth to a much greater degree than either single agent (FIG. 13B). In the OVCAR8 intraperitoneal xenograft model, Compound A or Compound B in combination with olaparib decreased formation of ascites. Moreover, the combination was well tolerated, with no significant weight loss compared to vehicle control (FIG. 14A). In addition, the OC316 (heterozygous BRCA2 mutated) ovarian cancer xenograft model was used to extend results observed with SKOv3 and OVCAR8 xenografts. Similar results were observed in the OC316 xenograft model (FIG. 13C and FIG. 14B). More importantly, the Compound B and olaparib combination prolonged survival compared to either agent alone, with tumor regression in 2 out of 10 xenografts (p<0.05) (FIG. 13C). To demonstrate relevance to breast cancer, xenografts with the BRCA-proficient TNBC cell line model MDA-MB-231 were studied. To reflect the original microenvironment, MDA-MB-231 cells were implanted directly into the mammary fat pad of female nude mice. One week after cell inoculation, mice were treated with single agent Compound B, olaparib, or the combination, and tumor volume was measured at the indicated intervals (FIG. 13D). Following treatment with either single agent Compound B or oalparib, tumor burden remained unchanged; however, the combination treatment inhibited tumor volume from day 28 and induced tumor regression in 5 of 10 mice (p<0.01) (FIG. 13D).

Tumors growing as xenografts were collected for histology with H&E and IHC staining. Routine H&E staining detected high-grade ovarian cancer in ovarian cancer xenograft models and breast cancer morphology in the breast cancer xenograft model, respectively. IHC of OVCAR8 and MDA-MB-231 xenograft tumors at study termination recapitulated in vitro studies. Compound B increased nuclear γ-H2AX staining, which was further increased by treatment with Compound B in combination with olaparib (p<0.0001) (FIG. 13E). Nuclear p-HDAC5 staining was decreased in Compound B treated tumors (p<0.0001), but not in olaparib treated tumors (FIG. 13E). These data are consistent with the notion that SIK2 inhibition enhances olaparib sensitivity through increasing nuclear localization of class IIa HDACs, decreasing MEF2D-mediated expression of DNA repair genes and increasing DNA damage. Taken together, these pre-clinical models demonstrate that SIK2 provides a novel target that could contribute to care of women with high-grade ovarian cancer and triple-negative breast cancer patients.

### Example 8—Discussion

The present study documents for the first time that inhibition of SIK2 synergistically enhances sensitivity of high grade serous ovarian and triple-negative breast cancers to PARP inhibitors in cell culture and xenograft models. Synergistic activity was noted in BRCA mutant and wild type cancers. A novel mechanism underlies this synergistic interaction. A decrease of PARP enzyme activity and phosphorylation of class-IIa HDAC 4/5/7 mediate the effects of SIK2 inhibitors on tumor cell growth in ovarian and breast cancers. They were also necessary and sufficient for the synergy observed between SIK2 inhibitors and PARPi. Inhibition of the phosphorylation of class-IIa HDAC 4/5/7 by Compound A or Compound B SIK2 inhibitor, 1) abolishes class-IIa HDAC 4/5/7-associated transcriptional activity of MEF2, 2) decreases MEF2D binding to regulatory regions with high-chromatin accessibility in DNA repair genes, and 3) represses the critical gene expression in DNA DSB repair pathway. Decreased expression of FANCD2, RAD51, and XRCC4 due to SIK2 inhibition likely contributes to PARPi sensitivity through a MEF2D-dependent mechanism.

SIK2 inhibition decreased phosphorylation of class-IIa HDACs and increased nuclear localization of class-IIa HDAC proteins. Phosphorylation of class-IIa HDACs controls their signaling-dependent nucleocytoplasmic shuttling. Under basal conditions, class-IIa HDACs are unphosphorylated and located in the nucleus, where they are recruited to their target genes through interaction with transcription factors, enabling their transcriptional repressive function. Class-IIa HDACs become phosphorylated in response to specific signals, leading to disruption of the interaction with transcription factors, their export to the cytoplasmic compartment, and de-repression of their targets. A member of Class-IIa HDACs was thought to be a component of the DNA damage response, recruited to the same dots, or repair foci, together with 53BP1 which is vital in promoting NHEJ. It was demonstrated that SIK2-rgulation of the MEF2D-mediated DNA repair pathway depends upon SIK2-mediated phosphorylation of Class-IIa HDACs. Thus, Class-IIa HDACs appear to be the key regulators of the synergy observed between SIK2 inhibitors and PARP inhibitors.

MEF2 transcription factors have a diversity of functions in a wide range of tissues and have been implicated in several diseases. The spectrum of genes regulated by MEF2 in different cell types depends upon extracellular signaling and on co-factor interactions that modulate MEF2 activity. The MEF2 domain is also involved in interactions with co-activators and co-repressors. Co-repressors that are thought to associate with the MEF2 domains of all MEF2 family proteins include the class IIa histone deacetylases HDAC4, -5, -7 and -9. According to the cBioPortal database, 6 to 21% of ovarian serous cystadenocarcinomas, invasive breast cancer, lung squamous cell and adenocarcinomas, uterine endometriod carcinomas, stomach adenocarcinomas, adrenocortical carcinomas, esophageal carcinomas, bladder urothelial carcinomas and pancreatic adenocarcinomas contain amplified MEF2 genes. The present study documents for the first time that MEF2 genes may act as oncogenes by regulating expression of genes involved in DNA DSB repair in ovarian and breast cancer. SIK2 inhibition decreased MEF2 gene promoter activity and repressed expression of critical genes in the DNA DSB repair pathway, supporting the notion that Compound A and Compound B enhance sensitivity to PARPi by decreasing MEF2's oncogenic function.

Synergetic interaction of SIK2 inhibitors and PARP inhibitors was observed with three structurally distinct PARP inhibitors (rucaparib, niraparib, and talazoparib) that have differential PARP trapping potential. Combinations of SIK2 inhibitors with PARP inhibitors of higher PARP trapping potential (Talazoparib) and with lower PARP trapping activity (olaparib) produced similar combination indexes, consistent with comparable synergy. Measurement of PARP enzyme activity indicated that the SIK2 inhibitors enhanced the effect of olaparib by further decreasing PARP enzyme activity in cancer cells with detectable PARP protein levels. Furthermore, 2 different SIK2i demonstrated synergy with PARPi, consistent with on-target effects of SIK2i. PARPi elicit significant responses in BRCA1 or BRCA2 mutation carriers with breast, ovarian, prostate, and pancreatic tumors. Thus, developing new strategies to enhance PARPi sensitivity and expand the utility of PARPi to DNA DSB repair competent tumors is crucial.

This study has a number of limitations. We have demonstrated that Olaparib-induced growth inhibition was significantly enhanced by combination treatment with either Compound A or Compound B in all 12 cancer cell lines tested, but not in non-tumorigenic ovarian and mammary epithelial cells. Although it was potentially due to different levels of replication stress and ongoing DNA damage between normal and malignant cells, this mechanism has not yet been confirmed and demonstrated functionally in the cell lines studied. It has been revealed that decreased expression of FANCD2, RAD51, and XRCC4 due to SIK2 inhibition likely contributes to PARPi sensitivity through a MEF2D-dependent mechanism, however, MEF2 regulates the expression of many molecules, there may be additional effects of MEF2D that contribute to sensitization to PARPi by in cooperation with downregulation of FANCD2, RAD51 and XRCC4. The in vivo data are strongly supportive of efficacy and low toxicity of SIK2i and PARPi combination in patients. Together, SIK2 inhibition decreases PARP enzyme activity and the expression of FANCD2, RAD51, and XRCC4, suggesting that the combination of SIK2i and PARPi has the potential to increase the magnitude and duration of PARPi activity in patients with different cancers. Thus, future clinical trials could be designed to determine whether the combination will benefit these patients. The present animal studies, particularly with Olaparib and Compound A/Compound B, did not show significant toxicity based on weight loss. The potential for tolerability in patients is further supported by the lack of synergism of the combination in the normal cell lines. PARP inhibitors are now approved for ovarian, breast, and prostate cancers. Compound B has exhibited minimal hematologic toxicity during toxicology studies and has been cleared by the FDA to initiate a phase I trial to find the maximum tolerated dose (MTD) of Compound B alone and in combination with paclitaxel in ovarian cancer. Assessing the combination of PARPi and SIK2i in the clinical setting should therefore be prioritized to optimize the use of these compounds and to maximize patient benefit.

### Example 9—Study Design

The objective of this study was to define the effect of SIK2i (Compound A and Compound B) on cancer cell growth in ovarian and triple-negative breast cancers, as well as to explore the synergy between SIK2 and PARP inhibition. It was demonstrated that SIK2 inhibition synergistically enhanced PARP inhibitor activity in a variety of ovarian and triple-negative breast cancer cell lines and xenograft models. In vitro experiments were performed in biological triplicate unless otherwise stated. Sample sizes were determined on the basis of previous experience and was sufficient to detect statistically significant differences between treatments. For in vivo experiments, mice were randomly assigned to treatment groups. Experiments were not blinded. Study groups were followed until individual tumor measurements reached 1.5 cm in diameter, at which point sacrifice was indicated in accordance with Institutional Animal Care and Use Committee protocols.

### Example 10—Statistical Analysis

Experiments were repeated two or three times. Data were plotted using GraphPad Prism 8 and compared using two-tailed student t test and one-way or two-way ANOVA test. Kaplan-Meier survival analysis of xenograft studies was performed using Log Rank test by GraphPad. Data are presented as Mean±STD unless specified. p<0.05 is considered significant. * p<0.05, ** p<0.01, *** p<0.001 and **** p<0.0001.

### Example 11—Cell Lines

Cell lines used in this study are listed in Table 1. The identity of all cell lines was confirmed with STR DNA fingerprinting in the MDACC Characterized Cell Line Core (supported by NCI P30CA016672). All cell lines were maintained in a 5% CO₂ incubator at 37° C. and mycoplasma tested with a Universal Mycoplasma Detection Kit from ATCC.

### Example 12—Viability Assays

Cell viability was determined using CellTiter-Glo® Luminescent Cell Viability Assay (Promega). 2000-4000 Cells were plated in 96-well plates and treated with a SIK2 inhibitor (Compound A or Compound B) and a PARP inhibitor (Rucaparib, Niraparib, olaparib or Talazoparib) alone or combined in serial dilutions 24 hrs after seeding. After 5-days of incubation, media were removed and a mixture of 30 uL of CellTiter-Glo reagent and 60 uL of culture media was added to each well. Luminescence was measured on a Synergy2 microplate reader (BioTek) after 10 min of shaking. Dose-response experiments were plotted and ICs( )values were calculated using nonlinear curve fitting with normalized response and variable slope by GraphPad Prism 8. Drug interaction of the two-drug combination using a constant ratio were processed and a Combination Index (CI) was calculated using CalcuSyn 2.0 (BIOSOFT). CI<1 indicates synergism, CI=1 indicates additive effect and CI>1 indicates antagonism.

### Example 13—Clonogenic Assays

Individual cells were seeded in 6-well plates in triplicate at the density of 200, 400 or 600 cells/well depending on doubling time. Cells were treated with single or double agents at different concentrations 1 day after seeding. Cells were grown up to two weeks until visible colonies were formed. Culture media with different treatments were refreshed every other day. At the conclusion of the experiment, cells were washed twice with PBS, fixed in 0.1% Brilliant Blue R with 10% v/v acetic acid and 30% v/v methanol for 1 min and washed with tap water until background was clear. Pictures were taken using a FluoChem E Imager. Clones with >50 cells were counted.

### Example 14—PARP Trapping Assay

Chromatin extraction was performed as described by Muvarak and colleagues using a subcellular protein fractionation kit (Thermo Scientific, 78840) (48). Briefly, Pellets were first lysed in membrane extraction buffer. Nuclei were then lysed in nuclear extraction buffer to isolate a nuclear soluble fraction. The remaining chromatin (nuclear insoluble) fraction was washed once with nuclear extraction buffer, then digested with 300 units of micrococcal nuclease to release chromatin-bound proteins. PARP binding in the chromatin fraction (indicative of PARP trapping) was assayed by Western blot analysis of the chromatin cell fraction against the PARP antibody.

### Example 15—PARP Enzyme Activity Assay

PARP enzyme activity assay. PARP enzyme activity was measured using a PARP universal colorimetric assay kit (R&D system, 4677-096-K). Cells were plated and treated with Compound A (6 μM)/Compound B (4 μM), Olaparib (0.05 μM), and a combination of both for 26 hrs on different ovarian cancer cell lines. Cell lysates were collected using cell extraction buffer. The biotinylated poly (ADP-ribose) deposited by PARP-1 in cell lysates onto immobilized histones in a 96-well plate was detected. Streptavidin-HRP (biotin-binding protein) and a colorimetric HRP substrate were added to produce relative absorbance that correlates with PARP-1 activity.

### Example 16—Chromatin Immunoprecipitation (ChIP) and RT-qPCR Analysis

OVCAR8, MDA-MB-231, SKOv3, OVCAR8-SIK2 KO or SKOv3-SIK2 KO cells (2 million) were cultured on a 150-cm plate, and treated the next day either with vehicle control or with Compound A (4 μM) or Compound B (5 μM) for 48 hrs. Chip assays were performed using the Magna Chip A Kit (Millipore). Briefly, cells after treatment with Compound A or Compound B for 48 hrs were incubated with 1% formaldehyde for 10 min at room temperature and neutralized with 1× glycine. Nuclei were isolated and sonicated to obtain 200-1000 bp DNA fragments using the QSONICA sonicator for 30 cycles with 10 seconds pulses at 100% amplitude with 2 min of incubation on ice between pulses. For individual ChIP assay, 100 μg of soluble chromatin per sample was immunoprecipitated with 8 μg of mouse IgG control antibody (Santa Cruz, sc-2025), 8 μg of rabbit control antibody (Millipore, PP64B), 8 μg of MEF2D antibody (Santa Cruz, sc-27115 3×), 8 μg of RNA polymerase II antibody (Abcam, ab817), 6 μg of Histone H3 (acetyl K27) antibody (Abcam, ab45173) or 6 μg of Histone H3 (tri methyl K4) (Abcam, ab8580). For ChIP-Sequence, 500 μg of chromatin per sample was immunoprecipitated with 40 μg of MEF2D antibody. Input determined from 1% of the cell lysate was used as a negative control. Purified and enriched DNA was quantified using real time quantitative PCR (RT-qPCR) with the following primers. FANCD2D Forward, 5′-ACC TGT TAT GAG CGT GAA GTC-3′ (SEQ ID NO:1) and Reverse, 5′-GAT GCA GGA CTG TGC ATT AGA-3′ (SEQ ID NO:2); EXD2 Forward, 5′-GGT CTG GCC TAA GGT TTC TTC-3′ (SEQ ID NO:3) and Reverse, 5′-CAG TTC ACG CTG GGT TCT T-3′ (SEQ ID NO:4); and XRCC Forward, 5′-GCA GTC TTC CTA GTC TCA ACT-3′ (SEQ ID NO:5) and Reverse, 5′-TTG CCC TTC TAG GAG CTT AAT G-3′ (SEQ ID NO:6). RT-qPCR was performed using iTaq Universal SYBR Green Supermix (Bio-Rad, 172-5124) in a CFX Connect RT-qPCR (Bio-Rad). Thermal cycling condition was as follows: 94° C. for 10 min, followed by 40 cycles of 94° C. for 20 sec, and 60° C. for 60 sec. Analysis of qPCR data was calculated using fold enrichment method (The ChIP signals are divided by the IgG antibody signals, 2^{-DDCt}).

### Example 17—ChIP-Sequence and Analysis

Sequencing was performed by the Sequencing and Microarray Facility (SMF) at MD Anderson Cancer Center. Briefly, Indexed libraries were prepared from 20 ng of Diagenode Biorupter sheared ChIP DNA using the KAPA Hyper Library Preparation Kit (Kapa Biosystems, Inc). Libraries were amplified by 8 cycles of PCR and then size distribution was assessed using the 4200 TapeStation High Sensitivity D1000 ScreenTape (Agilent Technologies) and quantified using the Qubit dsDNA HS Assay Kit (ThermoFisher). The indexed libraries were multiplexed, 10 libraries per pool. The pool was quantified by qPCR using the KAPA Library Quantification Kit (KAPA Biosystems) then sequenced on the Illumina NextSeq500 sequencer using the High-output 75 single read configuration. The raw reads were first preprocessed to remove sequencing adapters and low quality reads. The trimmed reads were then mapped to human reference genome hg19 using bowtie, with only uniquely mapped reads retained. The ChIP-seq occupancy profiles were generated by MACS 1.4 with the “-wig” parameter, and were normalized to 20 million total reads. Duplicated reads were automatically removed by MACS. ChIPseq peaks were called by MACS with p-value set to 1e-8. Peaks were annotated to associated genes according to their relative locations. T associated genes were identified as ChIP-seq target genes. Further functional analysis on these genes were carried out, including gene ontology (GO) analysis using DAVID. The enriched DNA binding motifs in ChIP-seq peak regions were identified and compared with known motifs using HOMER v4.8.

### Example 18—mRNA-Sequence and Analysis

Poly(A)-containing mRNA sequencing was performed by Sequencing and ncRNA program at MD Anderson cancer center. The indexed mRNA sequencing libraries were prepared from total RNA with RIN>9.0 using Illumina TrueSeq stranded mRNA library preparation kits (Illumina, RS-122-2101 and RS-122-2102), following guidance of an Illumina Truseq stranded mRNA protocol. In Brief, 200 ng of total RNA were used for poly(A) mRNA enrichment using oligo(dT) coated magnetic beads. The enriched and purified mRNA was fragmented into small pieces using divalent cations at elevated temperature. The cleaved RNA fragments were then reverse transcribed into first strand cDNA by reverse transcriptase using random hexamer primers for RT priming and reverse transcription, followed by second strand cDNA synthesis using DNA polymerase I and RNase H. These double strand cDNA fragments were end-repaired and then adenylated at 3′ ends with the addition of a single ‘A’ base to prevent self-ligation during subsequent ligation to the illumina index-specific adapters that has a single “T” at 3′ end which provides complementary overhang for ligating the adapter to the fragment. The raw library products were purified and enriched by PCR to create the final cDNA sequencing library. The indexed individual sequencing library was quantified using an Agilent Bioanalyzer High Sensitive DNA assay. To ensure the sufficient data coverage for high, medium and low copy transcripts, twelve indexed mRNA libraries were pooled and sequenced on an Illumina Nextseq 500 sequencer using TruSeq High Output Kit V2 150 cycles (FC-404-2001) in Paired-end E75 sequencing configuration. The raw data bcl files were de-multiplexed and converted into fastq file by using Illumina bcl2fastq2 conversion V 2.19 software (illumina). We used FastQC to perform a quality control of the FASTQ files and STAR (GRCh38, Gencode25 and STAR 2.6.1b) to map the reads against the reference genome and count the number of reads uniquely mapping to each gene, for each sample. Heatmap plots of selected genes showing their variation among different samples were generated in R, version 3.5.1, using the heatmap 2 function of g plots library. Public domain of gene pathways (qiagen.com/us/) was used to retrieve genes related to apoptosis and DNA Damage repair. Gene ontology enrichment analysis for differentially expressed genes was performed using the web-based tool Enrichr.

### Example 19—Immunoblot

Cells were incubated with and without treatment for the intervals indicated and then cells were incubated in lysis buffer (50 mM Hepes, pH 7.0, 150 mM NaCl, 1.5 mM MgCl₂, 1 mM EGTA, 10, 10% glycerol, 1% Triton X-100, 50 mM NaF, 1 mM Na₃VO₄ 1 mM PMSF, 10 μg/mL leupeptin and 10 μg/mL aprotinin) on ice for 30 min. Lysates were centrifuged at 15,000 g at 4° C. for 15 min, and supernatants were collected. To prepare subcellular fractions of nuclear soluble and chromatin-bound material, cells were treated with indicated drugs, and then cells were collected by scraping and subsequent centrifugation at 4° C. For fractionation, we used a Subcellular Protein Fractionation kit (Thermo Scientific, 78835) following the manufacturer's instructions. The protein concentration was assessed using a bicinchoninic acid (BCA) protein assay (Thermo Scientific, 23228). The proteins were separated by SDS-PAGE and transferred to Polyvinylidene difluoride (PVDF) membranes (Thermo Scientific, 88518). After being blocked with 5% BSA in TBST (tris-buffered saline with 0.1% tween 20 detergent), the membranes were incubated with primary antibodies at 4° C. overnight, followed by 1:2000 horseradish peroxidase (HRP)-conjugated secondary antibody (Thermo Scientific, anti-mouse 3439 and anti-rabbit 31463) for 40-60 min at room temperature. Bands were visualized using an ECL Western Blotting Substrate (PerkinElmer, NEL 104001EA). SIK2 (CST6919), p-HDAC4/5/7 (CST3443), HDAC5 (CST20458), HDAC4 (CST5392) and actin (CST4967) antibodies were purchased from Cell Signaling Technology. GAPDH (MAB374) antibody is from Millipore. PARP (551052) and MEF2D (610775) antibodies are from BD Pharmingen. Lamin A/C (sc-6215) antibody is Santa Cruz. Actinin (CBL-231) antibody is from Chemicon and α-Tubulin (T9026) antibody is from Sigma.

### Example 20—RNA Extraction and RT-qPCR Analysis

Cells were treated with and without Compound A or Compound B for 72 hrs and lysed in TRIzol (ThermoFisher, 15596026). Total RNA was extracted using an RNeasy kit (Qiagen, 217004) according to the manufacturer's instructions. cDNA was synthesized from 2 μg of RNA using the Superscript II First Strand Synthesis Kit (Invitrogen, 11904-018). RT-qPCR was performed using CFX Connect Real-time System (Bio-Rad) in a total volume of 20 μL, which included 10 μL of 2× SsoAdvanced Universal PCR master (PCR primers are included) and 5 ng of cDNA. Thermal cycling conditions were as follows: 95° C. for 2 min, followed by 40 cycles of 95° C. for 5 sec, and 60° C. for 30 sec. PrimePCR Custom Plates (96 well) which contain 2× SsoAdvanced Universal PCR master mix and PCR primers were custom ordered from Bio-Rad. Data were analyzed by the AACT method using GAPDH as a housekeeping gene. Experiments were run in triplicate.

### Example 21—Establishment of OVCAR8 and SKOv3 SIK2 CRISPR/Cas9 Knock Out Cell Lines

OVCAR8 and SKOv3 SIK2 knock out cell lines were established using CRISPR/Cas9 technology known in the art. Briefly, a plasmid with GFP containing Cas9 and the sgRNA expression were transfected to cancer cells. CRISPR-mediated knockout was performed using guide RNAs targeting exon 2 (AATAATCGATAAGTCTCAGC, SEQ ID NO:7) and exon 4 (GATTTTCAGCTTTGAGGTCA, SEQ ID NO:8). Transfected cells were isolated by FACS for single-cell culture 2-3 days after transfection, and then the cells were expanded and harvested for detection of the protein expression using western analysis.

### Example 22—Establishment of OVCAR8 and MDA-MB-231 MEF2D Inducible Cell Lines

OVCAR8 and MDA-MB-231 cells were infected with pLV(Exp)-Neo-CMV>tTS/rtTA_M2 lentivirus (VectorBuilder, VB160419-1020mes) and subsequently selected using 1 μg/mL of G418 according to the manufacturer's protocol (Dharmacon). Clonal populations were generated by limiting dilution under G418 (Corning 61-8833-100mg) selection. OVCAR8 and MDA-MB-231 cells with clonal population of CMV>tTS/rtTA were again infected with pLV(Tet)-EGFP:T2A:Puro-TRE-hMEF2D lentivirus (VectorBuilder, VB180504-1036gtn). Clonal populations were generated by limiting dilution under puromycin (Sigma, D-9897-1G) selection. Clones with the best expression efficiency were selected by western blotting under 1 μg/mL doxycycline (Sigma, D-9897-1G) for 48 hrs. OVCAR8-MEF2D and MDA-MB-231-MEF2D inducible cells were maintained in RPMI 1640 (Corning, 15-040-CV) supplemented with 10% FBS, G418 (1000 μg/mL for MDA-MB-231 and 500 μg/mL for OVCAR8) and puromycin (2 μg/mL for MDA-MB-231 and 1 μg/mL for OVCAR8).

### Example 23—RNA Interference

ON-TARGETplus pooled siRNAs targeting human HDAC4 (J-003497), HDAC5 (J-003498) and Non-targeting Control siRNA #2 (D-001810-02) and DharmaFect 4 (T-2004-03) were purchased from GE Dharmacon. 70 nM of siRNA and 0.2% DharmaFECT 4 were diluted in OPTI-MEM medium individually and then mixed together for 20 min at room temperature. Cells were then laid on top of siRNA-DharmaFECT mixture. Cells were lysed to determine target gene expression and prepared for luciferase activity assay 72 hrs post transfection (see Luciferase Reporter Assay below).

### Example 24—Immunohistochemical Staining (IHC)

Formalin fixed and paraffin embedded mouse tissue sections were deparaffinized and rehydrated in gradient ethanol solutions. Antigens were retrieved in Rodent Decloaker (BioCare Medical, RD913M) and microwaved twice in an EZ Retriever System V3 (BioGenex) at 95° C. for 5 min. Tissues were blocked in PeroxAbolish (BioCare Medical, PXA969M) for 30 min, Rodent Block M (BioCare Medical, RBM961L) for 30 min, and 5% BSA in PBS for 30 min Tissues were incubated with primary antibody as indicated overnight at 4° C. VisUCyte HRP Polymer IgG (R&D Systems, VC001-025 for mouse, VC003-025 for rabbit) was applied for 30 min at room temperature followed by DAB chromogenesis (BioCare Medical, BDB2004L). Tissues were counter-stained with CAT hematoxylin (Thermo Fisher, CATHE-M) for 20 sec. The slides were then dehydrated through gradient ethanol solutions and two passes of xylene and sealed with Permount (Thermo Fisher, SP15-100).

### Example 25—Luciferase Reporter Assay

MEF2 promoter activity was quantified using an MEF2 reporter assay Kit (QIAGEN, 336841 CCS-7024L). Cells were plated and after overnight incubation transfected with a mixture of a MEF2-responsive luciferase vector and a constitutively expressing Renilla luciferase vector (40:1) for 24 hrs. Cells were re-plated into a 96 well plate, incubated for 16 hrs and then treated with Compound A (4 μM) or Compound B (4 μM) for different intervals or with different doses of Compound A and Compound B for 24 hrs as indicated. Cells were then lysed for a dual luciferase assay. The relative luciferase activity of MEF2 was calculated by normalizing to Renilla luciferase activity. To quantify MEF2 promoter activity with and without knockdown of HDAC4 and HDAC5, cells were transfected with targeting siRNA or control siRNA for 24 hrs prior to transfection of a mixture of a MEF2-responsive luciferase and Renilla luciferase vectors. Cells were re-plated into a 96 well plate and then treated with Compound A (4 μM) or Compound B (4 μM) for 24 hrs. HDAC4 and HDAC5 siRNA knockdown efficiency was measured by western blot analysis.

### Example 26—Alkaline Single-Cell Agarose Gel Electrophoresis (Comet) Assays

1-2×10⁵ cells in 6-well plates were treated with DMSO, SIK2 inhibitor (Compound A and Compound B), olaparib or the combination of SIK2 inhibitor and olaparib. Treatment conditions were as follows: 1 μM of Compound A for HCC5032, OVCAR8 and SKOv3 and 0.5 μM of Compound A for MDA-MB-231 for 48 hrs; 5 μM of Compound B for all four cell lines for 48 hrs; and 5 μM of olaparib for all four cell lines for 16 hrs before harvest. Cells were trypsinized and resuspended at 2×10⁵/mL in cold PBS without Ca²⁺ and Mg²⁺. Cells were mixed with pre-warmed comet agarose at 1:10 (v/v) ratio. 10 uL of cell agarose mixture was plated onto comet slides pre-coated with 75 uL of agarose and chilled at 4° C. for 15 min to set. Cells were lysed in 25 mL of Lysis buffer at 4° C. for 2 hrs and washed with alkaline solution (pH 10). Comet slides were electrophoresed in cold alkaline solution at 20V for 15 min. Slides were rinsed with water and dried in 70% ethanol for 5 min Slides were then stained with Vista Green DNA dye and viewed using an Olympus epifluorescence microscope with a FITC filter. Images were captured using a 20× objective. 3-Well OxiSelect™ Comet Assay kit are from Cell Biolabs, Inc (STA-351). Experiments were run in triplicate and Olive Tail Moment was measured using CaspLab1.2.β2 software (CaspLab.com). Olive Tail Moment=Tail DNA%×Tail Length. 50-200 Cells were measured for each treatment and experiments were repeated twice independently to ensure reproducibility.

### Example 27—Immunofluorescence Staining

Cells on 22×22 mm coverslips were fixed in 4% formaldehyde in PBS (Thermo Fisher, J19943-K2) and permeabilized with 0.1% Triton X-100 (Sigma, X100) in PBS for 15 min. Cells were blocked with 5% BSA in PBS for 30 min and then stained with antibody overnight at 4° C., followed by secondary antibody and DAPI for 1 hr. Coverslips were mounted with Fluoro-Gel with TES buffer (Electron Microscopy Sciences, 50-246-96) and air dried. HDAC5 nuclear localization was evaluated by measuring nuclear fluorescence intensity of HDAC5. Cells were treated with DMSO, Compound A (3 μM) or Compound B (5 μM). After 24-hrs incubation, cells were fixed in 4% formaldehyde in PBS. Cells were stained as described above. Images were captured using an Olympus Model IX71 measuring nuclear HDAC4 fluorescence intensity in each cell using ImageJ (imagej.nih.gov/ij/). DNA damage visualized by γ-H2AX staining was evaluated by counting nuclear γ-H₂AX puncta in each cell. Cells were treated with DMSO, 1 μM of olaparib alone, 4 μM of Compound B or I μM of Compound A, or the combination of olaparib and SIK2 inhibitors. After 8 hrs incubation, cells were fixed in 4% formaldehyde in PBS. Cells were stained as described above. Images were captured using an Olympus IX71 microscope and nuclear γ-H₂AX puncta in each cells were counted using with Olympus CellSens Dimension software. HDAC5 (CST20458) and γ-H₂AX (CST2577) antibodies were purchased from Cell Signaling Technology. Experiments were repeated twice independently to ensure reproducibility and 50-200 cells were counted for each treatment.

### Example 28—Apoptosis

The percentage of apoptotic cells induced by Compound A/Compound B, olaparib, or a combination of both were measured on different ovarian cancer cell lines by fluorescence activated cell sorting (FACS) using FITC Annexin V/Dead cell Apoptosis Kit I (Thermo Fisher, cat. V13242) according to the manufacturer's instructions. Briefly, following indicated treatment, cells were harvested and washed once in 1×PBS. Afterward, cells were resuspended in 1× binding buffer containing 5 uL of fluorochrome-conjugated Annexin V plus 100 μg/ml PI (Propidium iodide) After 15 mins incubation at room temperature cells were centrifuged and resuspended in 200 μl 1× binding buffer and analyzed with flow cytometry. Stained cells were read on Gallios analyzer (Beckman Coulter) and 20,000 events were counted.

### Example 29—Growth of Human Ovarian and Breast Cancer Xenografts in Mice

Experiments with Hsd:Athymic nu/nu-Foxn1^{nu} mice (Envigo) were reviewed and approved by the Institutional Animal Care and Use Committee of M. D. Anderson Cancer Center (IACUC 00001052).

### Example 30—SKOv3 and OVCAR8 Ovarian Cancer Xenografts

Sixty female nu/nu mice were injected with 5×10⁶ SKOv3 cells subcutaneously or 3.5×10⁶ OVCAR8 cells intraperitoneally, respectively. After 7-days, mice were randomly assigned to the following treatment groups (n=10): 1) control vehicle, 2) Compound A (40 mg/kg for SKOv3 or 50 mg/kg for OVCAR8 per mouse, five times per week), 3) Compound B (40 mg/kg for SKOv3 or 50 mg/kg for OVCAR8 per mouse, five times per week), 4) olaparib (50 mg/kg per mouse, five times per week), 5) Compound A combined with olaparib, and 6) Compound B combined with olaparib. All mice were treated orally with vehicle control, single agent or combination of single agents for 4 weeks (SKOv3 xenograft models) or 6 weeks (OVCAR8 xenograft models) and sacrificed with CO₂ one week after completion of treatments. For SKOv3 xenograft models, tumors were measured every week in two dimensions using a digital caliper, and the tumor volume was calculated with the following formula: tumor volume (mm3)=0.5×ab² (a and b being the longest and the shortest diameters of the tumor, respectively). Mice were monitored until tumor burden reached 1500 mm3 (ethical endpoint). For OVCAR8 xenograft models, all tumors were weighed immediately after death.

### Example 31—O C316 Ovarian Cancer Xenografts

Forty female nu/nu mice were injected with 3.5×10⁶ cells intraperitoneally. After 7-day inoculation, tumor-bearing mice were randomly divided into 4 groups (n=10): 1) control vehicle, 2) Compound B (50 mg/kg five times per week), 3) olaparib (50 mg/kg per mouse, five times per week), 4) Compound B combined with olaparib, and 6) Compound B combined with olaparib. All mice were treated orally with vehicle control, single agent or combination of single agents for 5 week and then continually monitored for survival. Mice were monitored until dyspnea, weight loss, hunched posture, snuffling respiratory sounds or abdominal breathing were observed (ethical endpoint) for euthanasia.

### Example 32—MDA-MB-231 Breast Cancer Xenografts

Forty female nu/nu mice were injected with 0.8×10⁶ MDA-MB-231 cells into their fourth mammary fat pads. After 7-days, tumor-bearing mice were randomly divided into 4 groups (n=10): 1) control vehicle, 2) Compound B (50 mg/kg five times per week), 3) olaparib (50 mg/kg per mouse, five times per week), 4) Compound B combined with olaparib, and 6) Compound B combined with olaparib. All mice were treated orally with vehicle control, single agent or a combination of single agents for 5 weeks and then continually monitored for survival. Tumors were measured every week as noted above (SKOv3 xenograft models).

### Example 33

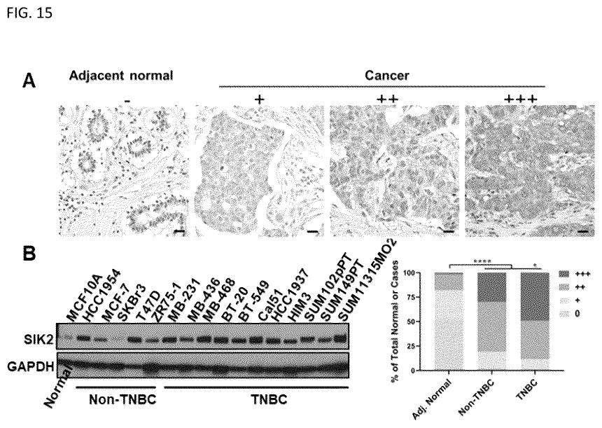

Expression of SIK2 in breast cancers was measured, performing immunohistochemical staining of a tissue microarray (TMA) with 120 non-TNBC cases, 130 TNBC cases and 61 normal and adjacent normal breast tissues. Intense (2-3+) SIK2 staining was observed in 80% of 120 non-TNBCs with lower levels of SIK2 protein (0-1+) in the remaining cancers, compared to intense (2-3+) SIK2 staining in 18% of adjacent normal breast tissue with lower levels of SIK2 protein (0-1+) in the remaining cases (FIG. 15A). More importantly, among 130 TNBCs, 88% exhibited intense staining with lower levels of SIK2 protein in the remaining cases. When SIK2 expression was measured in sixteen breast cancer cell lines, including eleven TNBC cell lines, SIK2 protein expression was significantly increased in the sixteen breast cancer cell lines compared to a normal breast cell line (MCF-10A). SIK2 was highly expressed in 11 of 11 TNBC cell lines (FIG. 15B).

### Example 34

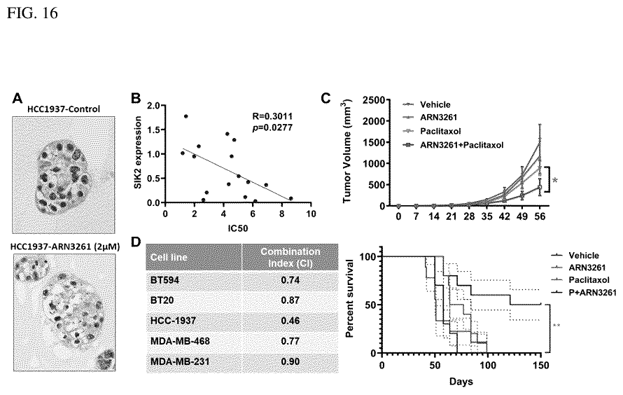

Compound B inhibits cell growth and increases paclitaxel sensitivity in breast cancer cells and xenografts. Growth inhibition was observed in a range of breast cancer cell lines after treatment with Compound B. Those breast cancer cell lines include MCF-7, ZR75-1, BT20, SKBr-3, AU565, MDA-MB-231, MDA-MB-468, MDA-MB-436, HCC1954, HCC1937, SUM1315M02, BT-549, SUM102PT, SUM149PT, HIM3 and Ca151. The IC50 of Compound B in these breast cancer cell lines ranged from 1.19 to 8.6 μM. Compound B inhibits organoid growth inducing cell death (FIG. 16A). The IC50 of Compound B is inversely correlated with SIK2 protein expression (FIG. 16B) measured by immunoblotting (FIG. 16B). Compound B also inhibited xenograft growth and prolonged the survival of mice bearing MDA-MB-231 orthotopic xenografts (FIG. 16C). To evaluate additive or synergistic interactions, a combination Index (CI value) was calculated with CalcuSyn software. Values less than 1 are considered synergistic and those equal to 1 are considered additive. At the combination index that reflected 90% inhibition, arguably the most relevant metric for cancer treatment, a combination of Compound B and paclitaxel exhibited synergy in 5/5 TNBC cell lines tested (FIG. 16D). These data support use of paclitaxel in combination with Compound B to achieve synergistic cytotoxicity for TNBC.

What is claimed is:

1. A method of increasing the magnitude and/or duration of activity of a poly (ADP-ribose) polymerase (PARP) inhibitor in a patient being treated with the PARP inhibitor, comprising administering to the patient in need thereof the PARP inhibitor and a salt-induced kinase 2 (SIK2) inhibitor.

2. A method of increasing the sensitivity of cancer cells to treatment with a PARP inhibitor, comprising contacting the cells with the PARP inhibitor and a SIK2 inhibitor.

3. A method of prolonging survival in a cancer patient in need thereof comprising administering to the patient in need thereof a PARP inhibitor and a SIK2 inhibitor.

4. A method of suppressing tumor growth in a cancer patient in need thereof, comprising administering to the patient in need thereof a PARP inhibitor and a SIK2 inhibitor.

5. A method of increasing the duration of remission for a cancer patient in need thereof comprising administering to the patient in need thereof a PARP inhibitor and a SIK2 inhibitor.

6. A method of preventing a relapse or reducing the incidence of relapse of a cancer patient in remission, the method comprising administering to the patient in need thereof a PARP inhibitor and a SIK2 inhibitor.

7. A method for reducing incidences of, or risk of, cancer recurrence in a patient deemed to be at risk of cancer recurrence, the method comprising administration to the subject of a PARP inhibitor and a SIK2 inhibitor.

8. The method of claim 7, wherein the subject deemed to be at risk of cancer recurrence is a subject who is in cancer remission.

9. A method of reducing hematologic toxicity during cancer treatment comprising administering to a patient in need thereof a PARP inhibitor and a SIK2 inhibitor.

10. The method of claim 9, wherein the hematologic toxicity comprises decreased white blood cells and decreased red blood cells.

11. A method of maintaining hematopoiesis during cancer treatment comprising administering to a patient in need thereof a PARP inhibitor and a SIK2 inhibitor.

12. A method of reducing toxicity and maintaining hematopoiesis during cancer treatment comprising administering to a patient in need thereof a PARP inhibitor and a SIK2 inhibitor.

13. A method of preventing hematologic side effects during cancer treatment comprising administering to a patient in need thereof a PARP inhibitor and a SIK2 inhibitor.

14. The method of any one of the preceding claims, wherein the cancer is ovarian or breast cancer.

15. The method of claim 14, wherein the ovarian cancer is primary or recurrent.

16. The method of claim 14, wherein the ovarian cancer is high-grade serous ovarian carcinoma (HGSOC).

17. The method of claim 14, wherein the SIK2 inhibitor inhibits growth of ovarian cancer cells.

18. The method of claim 14, wherein the PARP inhibitor is used to treat ovarian cancer and is selected from Olaparib, Rucaparib, and Niraparib.

19. The method of claim 14, wherein the breast cancer is triple-negative breast cancer.

20. The method of claim 17, wherein the triple-negative breast cancer comprises BRCA1/2 mutated breast cancer.

21. The method of claim 14, wherein the PARP inhibitor is used to treat breast cancer and is selected from Olaparib and Talazoparib.

22. The method of any one of the preceding claims, wherein the SIK2 inhibitor is Compound A or Compound B.

23. The method of any one of the preceding claims, wherein the SIK2 inhibitor is Compound B.

24. The method of any one of the preceding claims, wherein the SIK2 inhibitor is administered orally.

25. The method of any one of the preceding claims, wherein the SIK2 inhibitor blocks DNA double-strand break (DSB) repair in the cancer cells.

26. The method of claim 25, wherein the SIK2 inhibitor blocks DNA DSB repair by increasing nuclear localization of histone deacetylase (HDAC) 4/5, wherein the increased nuclear localization of HDAC4/5 blocks the activity of transcription factors associated with DNA DSB repair.

27. The method of claim 26, wherein the transcription factor associated with DNA DSB repair is a myocyte enhancer factor-2 (MEF2) protein.

28. The method of claim 27, wherein the MEF2 protein is MEF2D.

29. The method of any one of the preceding claims, wherein the combination of the PARP inhibitor and the SIK2 inhibitor induces increased levels of apoptosis in the cancer cells compared to cancer cells treated with only the PARP inhibitor or the SIK2 inhibitor.

30. The method of any one of the preceding claims, wherein the combination of the PARP inhibitor and the SIK2 inhibitor enhances sensitivity of the cancer cells to paclitaxel.

31. The method of any one of the preceding claims, wherein the PARP inhibitor is Olaparib.

32. The method of claim 31, wherein the SIK2 inhibitor sensitizes the ovarian or breast cancer cells to the PARP inhibitor by enhancing Olaparib-mediated inhibition of PARP enzyme activity.

33. The method of any one of the preceding claims, wherein the combination of the PARP inhibitor and the SIK2 inhibitor produces a synergistic growth inhibition of the cancer cells.

34. The method of any one of the preceding claims, wherein the combination of the PARP inhibitor and the SIK2 inhibitor decreases expression of one or more genes involved in regulation of DNA repair and apoptosis in the cancer cell compared to cells treated with Olaparib alone.

35. The method of claim 34, wherein the one or more genes involved in regulation of DNA repair and apoptosis in the cancer cell are selected from BRCA2, EXO1, FANCD2, LIG4, XRCC4, BAX, BCL2, CASP7, and TRADD.

36. The method of claim 35, wherein the one or more genes involved in regulation of DNA repair and apoptosis in the cancer cell are selected from EXO1, FANCD2, and XRCC4.

37. The method of claim 35 or 36, wherein expression of the one or more genes is decreased by decreasing MEF2D binding to promoter regions.
